(19) United States
(12) Patent Application Publication
(10) Pub. No.: US 2024/0344127 A1
(43) Pub. Date: Oct. 17, 2024

(54) ASYMMETRIC PROBE-ENHANCED LOOP-MEDIATED ISOTHERMAL AMPLIFICATION

(71) Applicant: CFD Research Corporation, Huntsville, AL (US)

(72) Inventors: David Edwin Gaddes, III, Madison, AL (US); Kyleigh Raye Jones, Huntsville, AL (US)

(21) Appl. No.: 18/632,071

(22) Filed: Apr. 10, 2024

Related U.S. Application Data
(60) Provisional application No. 63/495,401, filed on Apr. 11, 2023.
(60) Provisional application No. 63/501,322, filed on May 10, 2023.
(60) Provisional application No. 63/601,104, filed on Nov. 20, 2023.

(51) Int. Cl. C12Q 1/6876; C12Q 1/6853

(52) U.S. Cl. CPC C12Q 1/6876; C12Q 1/6853; C12Q 2600/16

(57) ABSTRACT

A method of performing an asymmetric loop-mediated isothermal amplification (LAMP) assay can include: providing the asymmetrical primer set of one of the embodiments; providing a reagent composition having a polymerase; combining the asymmetrical primer set and reagent composition with a sample having the target nucleic acid to form an amplification mixture; heating the amplification mixture to an amplification temperature within an amplification temperature range; maintaining the amplification temperature to amplify the target nucleic acid having the target sequence; and obtaining an amplified amount of the target nucleic acid having the target sequence. The asymmetrical primer set can include one primer of a primer pair to be at a lower (e.g., 10%) amount of the other primer.

### CROSS-REFERENCE TO RELATED APPLICATIONS

This patent application claims priority to U.S. Provisional Application No. 63/495,401 filed Apr. 11, 2023, U.S. Provisional Application No. 63/501,322 filed May 10, 2023, and U.S. Provisional Application No. 63/601,104 filed Nov. 20, 2023 which provisional applications are incorporated herein by specific reference in its entirety.

### U.S. GOVERNMENT RIGHTS

This invention was made with government support under W81XWH-22-C-0026 awarded by the Department of Defense. The government has certain rights in the invention.

### SEQUENCE LISTING

The instant application contains a Sequence Listing which has been submitted via Patent Center and is hereby incorporated by reference in its entirety. Said .xml copy, created on Apr. 8, 2024 is named C147810051US02, and is 107,170 bytes in size.

### BACKGROUND

### Field

The present disclosure relates to primers and/or methods of use in loop-mediated isothermal amplification (LAMP) assays, wherein the primers of a primer pair are present in asymmetric amounts.

### Description of Related Art

Precision medicine promises to improve many aspects of health and healthcare, making it possible for physicians to choose the best treatment and prevention strategy for each patient. Prior research has demonstrated that genomic and transcriptomic biomarkers are useful for disease diagnosis, prognosis, and monitoring, including cancers^{1,2}, cardiovascular health^{3,4}, depression^{5,6}, sepsis^{7,8}, and the like. Genetic variations and differences in gene expression levels have also been identified that affect pharmacokinetics (e.g., absorption, distribution, metabolism, elimination) and pharmacologic effects of specific drugs⁹. According to the FDA, serious side effects from therapeutics occur in more than 2 million people each year and cause more than 100,000 deaths. These interactions are due to individual variation in genes for drug-metabolizing enzymes, drug receptors, and drug transporters, leading to toxicity. In the same context, gene variation also causes adverse immune responses leading to death from common therapeutics, including antibiotics prescribed on the battlefield following traumatic injury to the soldiers.

With the advancement in genomic medicine, it is now possible to map the personalized pharmacogenomics response to limit/eliminate these adverse drug reactions.

However, in the currently followed process, patient samples are sent to centralized laboratories that utilize state-of-the-art qPCR and next-generation sequencing machines to detect the presence of gene variations and quantifying gene expression levels. The data then needs to be correctly interpreted to arrive at clinically actionable information. The typical turnaround times for these tests can span from a couple of days to several weeks. This long waiting time can be a matter of life and death if a drug with an adverse reaction gets administrated to the patients. Even though there has been development in portable PCR machines, these still need skilled staff, power and are not rugged for field use by the military.

Similarly, it can be medically important to identify the presence of an pathogen in a biological sample obtained from a patient. The sample tested by routine procedures can take too long considering the needs of the patient. Any delay of detecting a pathogen in the patient can severely impact the health of the patient.

Hence, there is a clear need to develop assays and instruments that can be utilized at the site to rapidly detect and quantify a specific set of genes, including single nucleotide polymorphism (SNP) or unique sequences, and their expression levels from a variety of different clinical samples.

### SUMMARY

In some embodiments, an asymmetrical primer set for amplifying a target nucleic acid can include an internal primer pair. The internal primer pair can include corresponding internal primers of: a forward internal primer having a forward hybridizing region on a 3′ end and a forward negative hybridizing region on a 5′ end (e.g., does not hybridize to target nucleic acid when hybridizing region hybridizes, but negative hybridizing sequence hybridizes to amplicon or other), wherein the forward hybridizing region hybridizes with a forward target sequence of the target nucleic acid and the forward negative hybridizing region does not hybridize with the target nucleic acid; and a backward internal primer having a backward hybridizing region on a 3′ end and a backward negative hybridizing region on a 5′ end, wherein the backward hybridizing region hybridizes with a complementary target sequence of a complementary target nucleic acid and the backward negative hybridizing region does not hybridize with the complementary target nucleic acid at the same time as the hybridizing region hybridizes to that target nucleic acid, wherein the complementary target nucleic acid is an amplicon of the target nucleic acid or a complementary strand of a double stranded nucleic acid. As configured, the forward hybridizing region hybridizes a target nucleic acid, while the negative hybridizing region does not hybridize to that same nucleic acid, but when the negative hybridizing region hybridizes with an amplicon it usually hybridizes with another region of the same nucleic acid strange (e.g., itself) to form a loop of hairpin or concatemer structure. The forward negative hybridizing region can be included into an amplicon to hybridize with another region of the amplicon to form the loop of the hairpin structure. Additionally, the backward negative hybridizing region (e.g., sequence thereof, or complement) can be included into an amplicon to hybridize with another region of the amplicon to form another loop of another hairpin structure In some aspects, a negative hybridizing region can be considered to be a negative region, where the hybridizing region is a positive region, respect to LAMP protocol. When the positive region hybridizes to a nucleic acid, the negative region does not hybridize with that nucleic acid. When the negative region hybridizes to a nucleic acid, the positive region does not hybridize with that nucleic acid.

In some embodiments, the asymmetrical primer set can include an outer primer pair comprising corresponding outer primers: a forward outer primer having a hybridizing region that hybridizes with the target nucleic acid at an outer region that is 3′ of where the forward hybridizing region hybridizes; and a backward outer primer having a hybridizing region that hybridizes with the complementary target nucleic acid at an outer region that is 3′ of where the backward hybridizing region hybridizes.

In some embodiments, the asymmetrical primer set includes at least one loop primer selected from a loop forward primer or a loop backward primer, where the at least one loop primer has a loop primer sequence with complementarity with the loop target sequence when in a loop of a hairpin amplicon or loop of a concatemer amplicon of the target nucleic acid. At least one of the forward internal primer or backward internal primers is present in an amount of at least 50% less than a corresponding primer in its primer pair.

In some embodiments, hybridizing between primers of the asymmetrical primer set and respective target sequences occurs in an aqueous medium with: a temperature below a melting point of the target nucleic acid (e.g., hybridizing nucleic acid) with each respective primer; a sodium concentration of from about 45 mM to about 55 mM; a magnesium concentration of from about 2 mM to about 8 mM; a GC content of the target sequence to be about 40% to about 65%; and a GC content of each primer of the asymmetric primer set to be about 40% to about 60%. In some aspects, the target sequence includes a single nucleotide polymorphism (SNP). However, a SNP is not required in the target sequence when sufficiently unique for selectivity for hybridization with the respective primer.

In some embodiments, the loop backward primer includes the sequence with complementarity with the target sequence. The complementarity and hybridization can be when the target sequence is on a loop of a hairpin amplicon or loop of a concatemer amplicon of the target nucleic acid.

In some embodiments, the loop forward primer includes the sequence with complementarity with the loop target sequence when the loop target sequence is in a loop of a hairpin amplicon or loop of a concatemer amplicon. In some aspects, the at least one loop primer includes a target-complementary sequence in a middle region that is complementary with a loop target sequence of a loop on a hairpin amplicon or loop of a concatemer amplicon of the target nucleic acid. In some aspects, the at least one loop primer includes a SNP-complementary sequence in a middle region that is complementary with the SNP sequence of a loop target sequence on a loop on a hairpin amplicon or concatemer amplicon of the target nucleic acid. In some aspects, the at least one loop primer includes a SNP-complementary sequence in a middle region, with the SNP-complementary sequence being at least 2, 3, 4, 5, 6, or 7 nucleotides from the 5′ end and at least 2, 3, 4, 5, 6, or 7 nucleotides from the 3′ end.

In some embodiments, the asymmetrical primer set can include: forward inner primer and backward inner primer having a length from about 38 nucleotides to about 42 nucleotides, the forward hybridizing region and backward hybridizing region independently being from about 20 nucleotides to about 22 nucleotides, and forward negative hybridizing region and backward negative hybridizing region being from about 18 nucleotides to about 20 nucleotides; forward outer primer and backward outer primer having a length from about 15 nucleotides to about 25 nucleotides; and at least one loop primer includes from about 11 nucleotides to about 25 nucleotides (nt).

In some embodiments, a reaction composition can include: the asymmetrical primer set of one of the embodiments; and a sample having the target nucleic acid, wherein the primer set hybridizes with the target nucleic acid or amplicon thereof. In some aspects, the target nucleic acid itself is an amplicon of another nucleic acid having the same sequence or complement thereof.

In some embodiments, a method of performing an asymmetric loop-mediated isothermal amplification (LAMP) assay can include: providing the asymmetrical primer set of one of the embodiments; providing a reagent composition having a polymerase; combining the asymmetrical primer set and reagent composition with a sample having the target nucleic acid to form an amplification mixture; heating the amplification mixture to an amplification temperature within an amplification temperature range; maintaining the amplification temperature to amplify the target nucleic acid having the target sequence; and obtaining an amplified amount of the target nucleic acid having the target sequence.

In some embodiments, the method can include: obtaining a biological sample having the target sequence of the target nucleic acid; purifying nucleic acids from the biological sample; and obtaining a purified nucleic acid sample as the sample that is combined with the asymmetrical primer set. In some aspects, the biological sample is from a mucosal swab, blood, urine, saliva, or other body fluid.

In some embodiments, the amplified target nucleic acid can be identified by a colorimetric assay with nucleic acid labeling dye. In some aspects, the method can include: visually analyzing a color generated by the amplified target nucleic acid over a period of time; and identifying a color that identifies presence of the target nucleic acid, and complement thereof, in the sample. In some aspects, the visualization can include: obtaining an image of the amplified target nucleic acid over the period of time; and analyzing the image for a color that indicates the presence of the target nucleic acid in the sample. Presence of the amplicons of the target nucleic acid indicates the presence of the original target nucleic acid in the sample.

In some embodiments, the methods can be used to determine a physiological condition of a subject, and providing the same. The physiological condition of the subject can be whether or not they have a disease or disorder (e.g., presence or absence of pathogen nucleic acid or nucleic acid of a genetic disease or disorder); identifying the target nucleic acid in the sample, wherein the sample is from a biological sample; and determining a physiological condition of a subject that provided the biological sample based on the presence of the target nucleic acid, wherein presence of the target nucleic acid indicates the physiological condition of the subject. However, disease states or conditions that are not related to pathogens, such as susceptibility to drug toxicity, may also be assessed with target nucleic acid sequences that are particular to that disease state or condition. Examples are provided herein for various types of target nucleic acids.

In some embodiments, the method can include: treating the amplification mixture with a fluorescent or colorimetric dye; assaying for fluorescent or colorimetric dye; and quantitating the amount of the target nucleic acid in the sample.

In some embodiments, the method is performed with heating to a temperature of between about 40° C. and about 75° C., between about 45° C. and about 70° C., or between about 50° C. and about 65° C.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A includes a schematic representation of a loop-mediated isothermal amplification (LAMP).

FIG. 1B includes a schematic representation of loop-mediated isothermal amplification (LAMP) with the primer set being configured such that the loop of the hairpin amplicons and concatemer amplicons excludes the target nucleic acid (e.g., SNP), which is on a linear portion of the hairpin or concatemer.

FIG. 1C includes a schematic representation of loop-mediated isothermal amplification (LAMP) with the primer set being configured such that the loop of the hairpin amplicons and concatemer amplicons includes the target nucleic acid (e.g., SNP) and where the forward inner primer and backward inner primer are present in an asymmetrical concentration with one being present in a higher concentration and one being present in a lower concentration, and where the backward loop primer is omitted.

FIG. 2A shows hairpin amplicon generation that is symmetric where both hairpin amplicons are generated in a substantially similar amount.

FIG. 2B shows hairpin amplification generation that is asymmetric wherein the hairpin that has the loop with the target sequence that is complementary to and hybridizes with the loop primer, where the forward inner primer is present at about 10% of the backward inner primer.

FIG. 3 includes a graph that shows the temperature dependence of the detection time of the target nucleic acid compared to the temperature maintained during the reaction and amplification.

FIGS. 4A and 4B include graphs that show the changes in detection time of one sequence (G) versus another sequence (A) with the loop probe for CYP2C19(G) versus CYP2C19(A).

FIGS. 5A and 5B include graphs that show the changes in detection time of one sequence (G) versus another sequence (A) with the loop probe for VKORC1(G) versus VKORC1(A).

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting.

Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology relates to a protocol for detecting the presence of a target nucleic acid sequence (e.g., a target nucleic acid) and related kits and systems for performance of the protocol. The protocol can be used as a method for target nucleic acid detection when the nucleic acid includes the target sequence. The protocol can be used to receive a sample on location, and then assay the sample for the presence of at least one target nucleic acid. The protocol can be performed in remote areas with a sample device that uses a colorimetric detector for identifying whether or not a sample has the target nucleic acid. The target nucleic acid can be obtained in a sample by any technique. In some aspects, the protocol can use a magnetic-bead-based nucleic acid extraction module, or other nucleic acid extraction system. The protocol can also use an isothermal nucleic acid amplification and a detection module to perform the detection of the target sequence, where complementary nucleic acids are used as primers and probes for amplification and detection purposes.

In some embodiments, the protocol can include bead-based nucleic acid extraction. A system can include a bead-based nucleic acid extraction module that will allow simple manual operation through a twist valve that allows sequentially connecting an internal microfluidic channel to different buffer reservoirs to extract nucleic acids from different clinical samples. Different versions of pre-filled modules can be fabricated based on the target of interest (DNA, RNA) and clinical sample choice (e.g., buccal swab, blood, urine, saliva). However, any nucleic acid extraction system or protocol can be used to obtain nucleic acids. In some aspects, the nucleic acid extraction results in an amount of at least 10 copies per microliter of the nucleic acids of interest.

In some embodiments, the nucleic acid amplification and detection module can be configured as a cartridge, which can include multiple reservoirs pre-loaded with lyophilized primers, amplification reagents, and nucleic acid labeling dyes for implementing loop-mediated isothermal amplification (LAMP) assays. In some aspects, the protocol and cartridge achieve the sample heating and temperature required for LAMP amplification using exothermic chemical reactions, which require no power for operation. In other aspects, a battery or connected power source can be used to run heaters to heat the sample to the temperatures described herein. Additionally, the cartridge can allow for colorimetric detection, where the color indicates a confirmation for the presence of the target nucleic acid. For example, a positive color can indicate the presence of a gene variant which is detected. The presence of the target nucleic acid can be visually obtained based on a colorimetric change. No color indicates the absence of the target nucleic acid. Additionally, the data can be analyzed by a simple imaging setup (e.g., camera, such as on a smart phone or other) coupled with low-power LEDs and appropriate filters that can allow for quantification of gene expression levels. Examples of such cartridges and systems that can be used with the present protocol include U.S. Provisional Application No. 63/501,322, and U.S. Provisional Application No. 63/601,104.

In some embodiments, the timing of the generation of the color for visualization provides an indication of the presence of the target sequence. For example, due to amplification effects, even samples without the target sequence can generate non-target nucleic acids to generate a color over time. As such, a color determination for the target sequence can be within a defined time range or before a cutoff threshold time, and any color outside the range or after the threshold is not considered a positive result.

### Background of Lamp

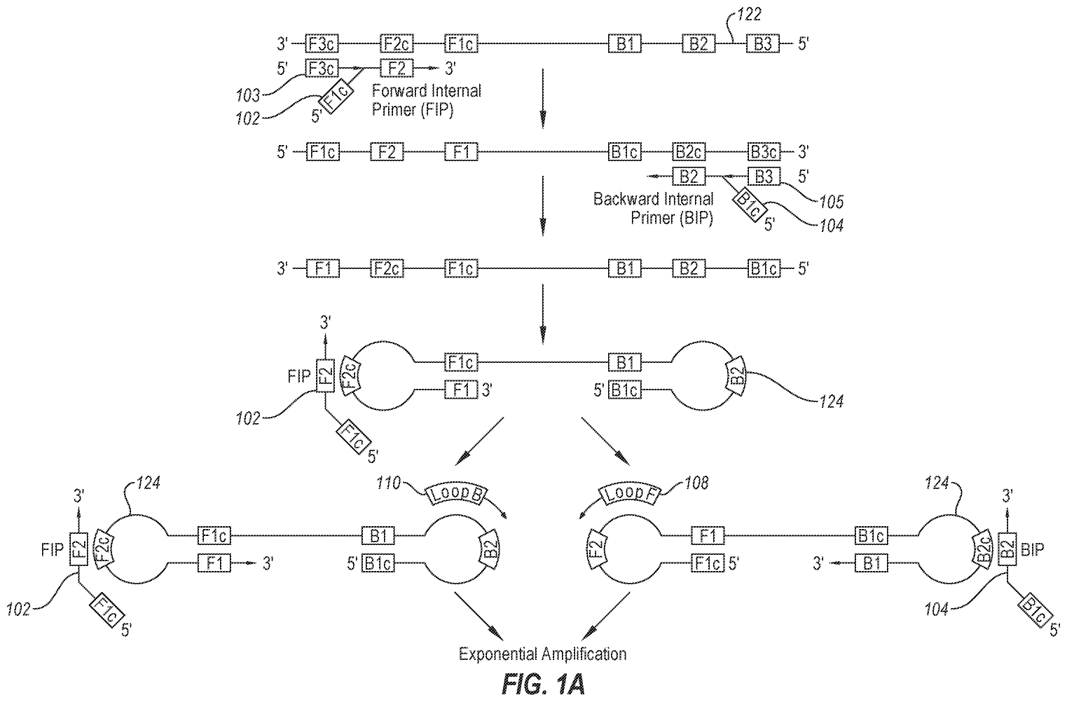

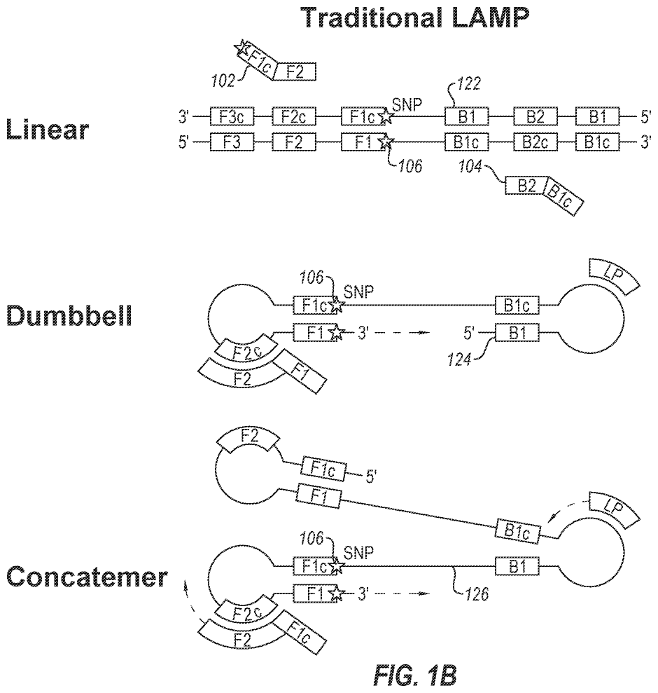

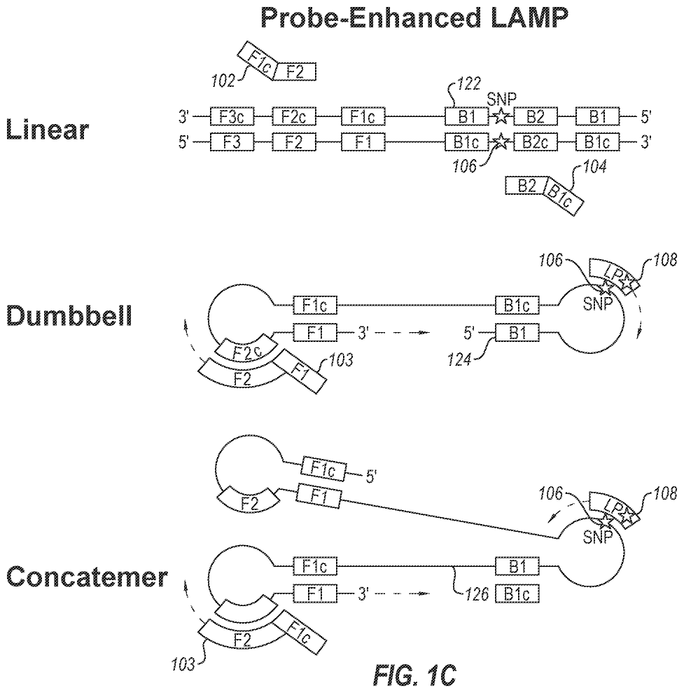

Loop-mediated isothermal amplification (LAMP or Loopamp) is an isothermal DNA amplification procedure using a set of four to six primers, two to three “forward” and two to three “reverse” that specifically recognize the target DNA. See Nagamine et al., Nucleic Acids Res. (2000) 28:e63; Nagamine et al., Clin. Chem. (2001) 47:1742-43; U.S. Pat. No. 6,410,278; U.S. Patent Appl. Nos. 2006/0141452; 2004/0038253; 2003/0207292; and 2003/0129632; and EP Patent Appl. No. 1,231,281. Briefly, one set of primers are designed such that approximately ½ of the primer is positive strand the other ½ of the primer sequence is negative strand. After strand displacement amplification by the polymerase, a nucleic acid structure that has hairpin loops on each side is created. From this structure, repeating rounds of amplification occur, generating various sized product. A by-product of this amplification is the formation of magnesium-pyrophosphate, which forms a white precipitate leading to a turbid reaction solution. This presence of turbidity signifies a positive reaction while the absence of turbidity is a negative reaction. Additional additives, such as calcein, allow other visualizations to occur; as for calcein it enables fluorescence detection. See FIGS. 1A-1C. The amplification reaction occurs under isothermal conditions (at approximately 65° C.) and continues with an accumulation of 109 copies of target in less than an hour. LAMP can be modified so that the SNP or target sequence of the loop probe is on the loop, as shown in FIG. 1C, which is defined as probe-enhanced LAMP (PE-LAMP).

The amplification product can be detected via photometry, measuring the turbidity caused by magnesium pyrophosphate precipitate in solution as a byproduct of amplification. This can allows visualization by the eye, camera, or via simple photometric detection approaches for small volumes. The reaction can be followed in real-time either by measuring the turbidity or by fluorescence using intercalating dyes, such as SYTO 9. Dyes, such as SYBR green, can be used to create a visible color change that can be seen with the naked eye without the need for expensive equipment, or for a response that can more accurately be measured by instrumentation. Dye molecules intercalate or directly label the DNA, and in turn can be correlated with the number of copies initially present. Thus, colorimetry can be used to visualize, with eye or camera, the presence or absence of the target nucleic acid. The color shows when the target nucleic acid is present, and the color is absent when the target nucleic acid is absent.

Accordingly, the PE-LAMP can be performed so that the sequence of the target and sequences of the primers are configured to generate the hairpin loop with the SNP or other target sequence on the loop structure. That is, the target sequence can be selected, and the primer set can be created to have sequences to generate the hairpin structure with the SNP or other target sequence after the strand displacement amplification. As such, all of the primers are designed for causing strand displacement amplification to generate the hairpin structure with the SNP or target sequence on the loop. This amplification system then has the loop primer having the complementary sequence to hybridize with the target sequence with the SNP or other target sequence when on the loop of the hairpin or concatemer.

The size of the loops of the hairpin structure or concatemer can be tailored to be between about 25 nucleotides (nt) and about 75 nt, about 30 nt to about 60 nt, about 40 nt to about 50 nt, or about 45 nt.

As defined herein, one of the primers of a primer pair is included in an asymmetrical amount compared to the companion primer of the primer pair. This asymmetrical amount of a primer pair causes one primer to be in a higher amount and the other primer to be in a lower amount, comparatively to each other. Any of the primer sets can be configured with asymmetric amounts as descried herein. This version can be referred to as asymmetric probe enhanced loop-mediated isothermal amplification (APE-LAMP). For example, one of the forward inner primer (FIP) or backward inner primer (BIP) can be included in a lesser amount compared to the other inner primer, such as at least half (50%), a quarter (25%), 10%, 15%, 10%, 5%, or 1% of the other. In a more preferred embodiment, the lesser primer of the primer pair can be from about 5% to about 15%, about 8% to about 13%, or about 10% to about 12%, or about 8% to about 12% or about 9% to about 11%, or about 10% of the other.

In some embodiments, the primer pair having the asymmetrical amount is the forward outer primer (F3) or backward outer primer (B3), which can present at the same relative amounts as described above.

In some embodiments, the primer pair having the asymmetrical amount is the forward loop primer (Loop F) or backward loop primer (Loop B), which can present at the same relative amounts as described above.

In a preferred embodiment, forward inner primer (FIP) or backward inner primer (BIP) includes an asymmetric amount and the forward loop primer (Loop F) or backward loop primer (Loop B) includes an asymmetric amount. In another preferred embodiment, forward inner primer (FIP) or backward inner primer (BIP) includes an asymmetric amount and the forward loop primer (Loop F) includes a typical amount while the backward loop primer (Loop B) is excluded (or at a non-functional amount). In another preferred embodiment, forward inner primer (FIP) or backward inner primer (BIP) includes an asymmetric amount and the backward loop primer (Loop B) is present while the forward loop primer (Loop F) is excluded. In another preferred embodiment, the forward inner primer (FIP) is present at a lesser amount (e.g., about 10%) of the backward inner primer (BIP, about 100%).

In one aspect, provided herein is a reagent preparation for loop-mediated isothermal amplification of nucleic acids comprising: at least one polymerase enzyme, wherein the enzyme is capable of strand displacement, a target-specific primer set, and dinucleotide triphosphates (dNTPs); wherein said reagent preparation is water soluble and stable above 4° C. In some embodiments, the polymerase enzyme capable of strand displacement is Bst enzyme. If the target is RNA, the reagent preparation also includes a reverse transcriptase. In some embodiments, the reverse transcriptase is AMV reverse transcriptase.

In another aspect, provided herein is a method of making a reagent preparation for loop-mediated isothermal amplification of nucleic acids comprising the steps of: (a) providing a buffered aqueous solution of (1) at least one polymerase enzyme, (2) a target-specific primer set, (3) dinucleotide triphosphates (dNTPs); and (b) drying the solution to form the reagent preparation; wherein the reagent preparation is water soluble and is stable above 4° C. If the target is RNA, the method further includes a reverse transcriptase. In some embodiments, the reverse transcriptase is AMV reverse transcriptase.

Any suitable DNA polymerase capable of strand displacement can be employed. As used herein, the term “strand displacement” refers to the ability of the enzyme to separate the DNA strands in a double-stranded DNA molecule during primer-initiated synthesis. The enzyme can be a complete enzyme or a biologically active fragment thereof. The enzyme can be isolated and purified or recombinant. In some embodiments, the enzyme is thermostable. Such an enzyme is stable at elevated temperatures (>40° C.) and heat resistant to the extent that it effectively polymerizes DNA at the temperature employed. Sometimes the enzyme can be only the active portion of the polymerase molecule, e.g., Bst large fragment. Exemplary polymerases include, but are not limited to Bst DNA polymerase, Vent DNA polymerase, Vent (exo-) DNA polymerase, Deep Vent DNA polymerase, Deep Vent (exo-) DNA polymerase, Bca (exo-) DNA polymerase, DNA polymerase I Klenow fragment, (D29 phage DNA polymerase, Z-Taq™ DNA polymerase, ThermoPhi polymerase, 9° Nm DNA polymerase, and KOD DNA polymerase. See, e.g., U.S. Pat. Nos. 5,814,506; 5,210,036; 5,500,363; 5,352,778; and 5,834,285; Nishioka, M., et al. (2001) J. Biotechnol. 88, 141; Takagi, M., et al. (1997) Appl. Environ. Microbiol. 63, 4504. In some examples, about 1 to 20 U (such as about 1 to 15 U, about 2 to 12 U, about 10 to 20 U, about 2 to 10 U, or about 5 to 10 U) of DNA polymerase is included in the reaction. In some examples, the polymerase has strand displacement activity and lacks 5′-3′ exonuclease activity. In one non-limiting example, the DNA polymerase is Bst 2.0 WarmStart™ DNA polymerase (New England Biolabs, Ipswich, Mass.), for example about 16 U Bst 2.0 WarmStart™ DNA polymerase per reaction.

If the target nucleotide is RNA, any suitable reverse transcriptase may be employed. In some embodiments, the reverse transcriptase is thermostable. Exemplary examples of reverse transcriptases used to convert an RNA target to DNA may include, but are not limited to Avian Myeloblastosis Virus (AMV) reverse transcriptase, Moloney Murine Leukemia Virus (M-MuLV, MMLV, M-MLV) reverse transcriptase, MonsterScript reverse transcriptase, AffinityScript reverse transcriptase, Accuscript reverse transcriptase, StrataScript 5.0 reverse transcriptase 5.0, ImProm-II reverse transcriptase, Thermoscript reverse transcriptase and Thermo-X reverse transcriptase and any genetically altered forms or variants of the aforementioned reverse transcriptases. In some examples, about 0.1 to 50 U (such as about 0.2 to 40 U, about 0.5 to 20 U, about 1 to 10 U, or about 2 to 5 U) of RT is included in the reaction. In a particular non-limiting example, the reaction includes 2 U/reaction of AMV RT.

The buffered aqueous solution suitable for the compositions and methods provided herein are those that permit the desired activity of the nucleic acid synthesizing enzyme. Glycerol is typically a component of buffered aqueous solutions for enzymes and acts as a stabilizing agent. In one embodiment, the aqueous buffer comprises 25 mM Tris-HCl pH 8.8, 12.5 mM KCl, 10 mM MgSO₄, 12.5 mM (NH₄)₂SO₄, and 0.125% Tween 20. In some embodiments, an agent that facilitates melting of the DNA is also included. Exemplary agents that facilitate the melting of DNA include but are not limited to betaine, trehalose, tetramethylone sulfoxide, homoectoine, 2-pyrrolidone, sulfolane, and methyl sulfone.

Primers, as used herein, are short nucleic acids, generally DNA oligonucleotides 10 nucleotides or more in length, such as 10-60, 15-50, 20-45, or 20-40 nucleotides (nt) in length (e.g., 38 to 42 nt). Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, such as by the APE-LAMP, RT-APE-LAMP, or other nucleic acid amplification methods known in the art.

The primers can have complementarity with the target sequence of the target nucleic acid, complement thereof, and amplicons thereof. That is, the primer has a sequence that is complementary to and hybridizes with the target sequence of the target nucleic acid, complement of the target nucleic acid, or amplicons thereof. The hybridizing can be under the hybridization conditions described herein below the melting temperature. The primer may have complementarity with the respective target sequence of at least about 80%, at least about 85%, at least about 90%, at least about 95%, at least about 99%, or 100% complementarity.

The reaction mixture, including sample, APE-LAMP primers, buffers, nucleotides, DNA polymerase, optionally reverse transcriptase, and any other components, is incubated for a period of time and at a temperature sufficient for production of an amplification product. In some examples, the reaction conditions include incubating the reaction mixture at about 37° C. to about 70° C. (such as about 40° C.-70°, about 50° C.-65° C., or about 45° C.-60° C.), for example about 40° C., about 45° C., about 50° C., about 55° C., about 60° C., about 65° C., or about 70° C. The reaction mixture is incubated for at least about 5 minutes (such as about 10, about 15, about 20, about 30, about 40, about 50, about 60, about 70, about 80 about 90, about 100, about 110, about 120 minutes or more), for example about 10-120 minutes, about 15-90 minutes, about 20-70 minutes, about 45-75 minutes, or about 30-60 minutes. In one non-limiting example, the reaction conditions include incubating the reaction mixture at about 65° C. for about 60 minutes. The reaction can optionally be terminated by incubation for a short time at a higher temperature, for example about 2-10 minutes at about 80° C. In some examples, the assay is performed using a chemical heater device to maintain the sample at the reaction temperature for a period of time (see, e.g., Singleton et al., Proc. SPIE 8615:8625oR, 2013; Curtis et al., PLoS ONE 7:e31432, 2012). The temperature can be maintained by an electronic heating device powered by batteries or via a power cord.

Several LAMP-based techniques have been developed for allele-specific amplification.

FIGS. 1A-1C show examples of loop-mediated isothermal amplification (LAMP), which can be used for a nucleic acid amplification test (NAAT) that does not require thermocycling, unlike traditional PCR. A typical reaction involves incubating the LAMP mixture to 60° C.-65° C. for 15 to 60 minutes. In comparison to PCR which requires cycles of temperatures from 55° C.-95° C. requiring a total time of at least 2 hours^{10, 11}. Further, isothermal amplification in LAMP requires less complex equipment than traditional PCR and can operate at the point of care without specialized equipment or power requirements.

In some embodiments, the LAMP can use 6 primers. In other embodiments, the asymmetrical probe enhanced LAMP (APE-LAMP) can be performed with only 5 primers, where only one loop primer, either forward or backwards, is used instead of two loop primers. However, all 6 primers can be used with APE-LAMP if desired, especially with asymmetry in the relevant amount of the primer pairs. The six LAMP primers are the: forward internal primer (FIP) 102, backward internal primer (BIP) 104, forward outer primer (F3) 103, backward outer primer (B3) 105, loop forward (LF) 108, and loop backward (LB) 110, which are shown in the FIGS. 1A-1C. The amplification process yields concatemers that extend from their 3′ end to form longer concatemers. Moreover, the FIP 102, the BIP 104, or the loop primers 108,110 can hybridize to the loop of the concatemer to generate new concatemers. These multiple pathways for amplification in LAMP result in a more rapid process than in PCR.

FIG. 1A shows the target nucleic acid 122 including an F3c region at a 3′ end, a F2c region downstream of the F3c region, and a F1c region downstream of the F2c region. The FIP 102 includes an F2 region that is complementary to and hybridizes with the F2c region of the target nucleic acid 122. At the 5′ end, the target nucleic acid 122 includes a B3 region, a B2 region upstream of the B3 region, and a B1 region upstream of the B2 region. The F2 region of the FIP 102 hybridizes with the F2c region of the target nucleic acid 122. The forward outer primer (F3) hybridizes with the F3c region of the target nucleic acid 122, which can result in a compliment of the target nucleic acid being generated.

The compliment of the target nucleic acid includes the F1c region at the 5′ end, a F2 region downstream of the F1c region, and a F1 region downstream of the F2 region, and the 3′ end includes the B3c region, the B2c region upstream of the B3c region, and the B1c region upstream of the B2c region. The backward inner primer includes the B2 region that hybridizes with the B2c region of the complimentary nucleic acid. The backward outer primer (B3) hybridizes with the B3c region of the complementary nucleic acid. The BIP then generates the amplicon that can fold into the hairpin having the loop.

The loop is formed by the terminal F1 region on the amplicon hybridizing with the F1c region on the amplicon, and the terminal B1c region of the amplicon hybridizing with the B1 region of the amplicon. This leaves the B2 region as a loop between the hybridized B1 region and B1c region, and the F2c region as a loop between the hybridized F1 region and the F1c region.

In some instance the BIP 104 hybridizes with a loop having the B2c region and the Loop F primer hybridizes with the loop having the F2 region. In other instances, the FIP 102 hybridizes with the loop having the F2c region and the Loop B primer hybridizes wit the loop having the B2 region.

The traditional approach involves designing a forward inner primer (FIP) 102 or backward inner primer (BIP) 104 with a base pair on the 5′ end of the target nucleic acid 122 that matches the SNP of interest (SNP, 106) (or other unique target sequence with unique sequence for specificity), as shown in FIG. 1B (Traditional LAMP). Theoretically, if the SNP of interest is not present, extension from the 3′ site of the dumbbell 124 (e.g., hairpin) and concatemer structures 126 will not occur. However, a 3′ mismatch is often ignored in isothermal amplification, and surprisingly, the modified primers often performed comparably to their unmodified counterparts in some cases and worse in others¹². To address these issues, a newer technique called probe-enhanced LAMP, or PE-LAMP as shown in FIG. 1C, can circumvent this issue by moving the SNP 106 to the loop portion of the concatemer 126.¹³ This process differs from traditional LAMP in that the loop primer 108 or 110 (LP) is used to differentiate the SNP 106 rather than the FIP 102 or BIP 104.

In some embodiments, APE-LAMP is a modification of the LAMP protocol where the SNP 106 is moved to the loop portion of the concatemer 126, as shown in FIG. 1C (Probe-Enhanced LAMP—PE-LAMP), and where one primer of one or more primer sets is present in an asymmetric amount. APE-LAMP uses the loop primer 108 (LP) to differentiate the SNP 106, and the site of interest is designed at the center of a short LP 108, typically 11-15 bp long. Moreover, the forward and backward inner primers (103, 105, respectively) do not have symmetric concentrations. This approach aims to prevent the generation of additional concatemers rather than preventing the extension of concatemers, as in traditional LAMP. Moreover, designing LAMP primers for SNP detection is challenging, and traditional LAMP primer designs often have limited potential designs. PE-LAMP, on the other hand, offers more design possibilities.

Additionally, APE-LAMP can include only one loop primer, whether the forward loop primer or the backward loop primer. Therefore the loop primers are also present in an asymmetric amount with respect to each other.

While FIG. 1B shows the traditional LAMP detection strategy, FIG. 1C shows the probe-enhanced LAMP detection strategy. The asymmetrical primer concentrations protocol can be used for either the traditional lamp or PE-LAMP. In these figures, the SNPs 106 are labeled with a light star, and the complementary base pair for SNP detection is labeled with a darker star on the loop forward primer (LFP) 108. The traditional strategy places the SNP or target sequence of interest on the 3′ end of either the FIP or BIP. In contrast, PE-LAMP places the SNP or target sequence of interest at the center of the loop of the hairpin of a dumbbell or concatemer, such that the loop primer (LP) hybridizes with the loop.

With reference to FIG. 1C as an example to illustrate the elements of the APE-LAMP primers, it is noted that the primers can have variations in length (e.g., number of nucleotides), GC content, and melting temperature. By defining these parameters, the particular APE-LAMP primers can be generated for being able to amplify and detect a target sequence.

In some embodiments, the forward internal primer (FIP) 102 can include two regions as shown, which are the F1c region at 5′ end and the F2 region at 3′ end. The F1c region can have a length of from about 18 nt to about 24 nt, about 19 nt to about 23 nt, about 20 nt to about 22 nt, or about 21 nt. The F2 region can have a length of from about 16 nt to about 22 nt, about 17 nt to about 21 nt, about 18 nt to about 20 nt, or about 19 nt.

In some embodiments, the backward internal primer (BIP) 104 can include two regions as shown, which are the B1c region at 5′ end and the B2 region at 3′ end. The B1c region can have a length of from about 18 nt to about 24 nt, about 19 nt to about 23 nt, about 20 nt to about 22 nt, or about 21 nt. The B2 region can have a length of from about 16 nt to about 22 nt, about 17 nt to about 21 nt, about 18 nt to about 20 nt, or about 19 nt.

The forward outer primer (F3) 103 can include a length of from about 11 nt to about 29 nt, about 12 nt to about 28 nt, about 13 nt to about 27 nt, about 14 nt to about 26 nt, about 15 nt to about 25 nt, about 17 nt to about 23 nt, or about 19 nt to about 20 nt.

The backward outer primer (B3) 105 can include a length of from about 11 nt to about 29 nt, about 12 nt to about 28 nt, about 13 nt to about 27 nt, about 14 nt to about 26 nt, about 15 nt to about 25 nt, about 17 nt to about 23 nt, or about 19 nt to about 20 nt.

The forward loop primer (FL) 108 can include a length of from about 7 nt to about 29 nt, about 10 nt to about 28 nt, about 13 nt to about 27 nt, about 14 nt to about 26 nt, about 15 nt to about 25 nt, about 17 nt to about 23 nt, or about 19 nt to about 20 nt.

The backward loop primer (BL) 110 can include a length of from about 7 nt to about 29 nt, about 10 nt to about 28 nt, about 13 nt to about 27 nt, about 14 nt to about 26 nt, about 15 nt to about 25 nt, about 17 nt to about 23 nt, or about 19 nt to about 20 nt.

In some embodiments, the forward internal primer (FIP) 102 can include two regions as shown, which are the F1c region at 5′ end and the F2 region at 3′ end. The F1c region can have a GC content of from about 40% to about 65%, about 41% to about 64%, about 42% to about 63%, about 43% to about 62%, about 44% to about 61%, about 45% to about 60%, or about 50% to about 55%. The F2 region can have a GC content of from about 40% to about 65%, about 41% to about 64%, about 42% to about 63%, about 43% to about 62%, about 44% to about 61%, about 45% to about 60%, or about 50% to about 55%.

In some embodiments, the backward internal primer (BIP) 104 can include two regions as shown, which are the B1c region at 5′ end and the B2 region at 3′ end. The B1c region can have a GC content of from about 40% to about 65%, about 41% to about 64%, about 42% to about 63%, about 43% to about 62%, about 44% to about 61%, about 45% to about 60%, or about 50% to about 55%. The B2 region can have a GC content of from about 40% to about 65%, about 41% to about 64%, about 42% to about 63%, about 43% to about 62%, about 44% to about 61%, about 45% to about 60%, or about 50% to about 55%.

The forward outer primer (F3) 103 can include a GC content of from about 40% to about 65%, about 41% to about 64%, about 42% to about 63%, about 43% to about 62%, about 44% to about 61%, about 45% to about 60%, or about 50% to about 55%.

The backward outer primer (B3) 105 can include a GC content of from about 40% to about 65%, about 41% to about 64%, about 42% to about 63%, about 43% to about 62%, about 44% to about 61%, about 45% to about 60%, or about 50% to about 55%.

The forward loop primer (FL) 108 can include a GC content of from about 35% to about 70%, about 40% to about 65%, about 43% to about 63%, about 43% to about 62%, about 44% to about 61%, about 45% to about 60%, or about 50% to about 55%.

The backward loop primer (BL) 110 can include a GC content of from about 35% to about 70%, about 40% to about 65%, about 43% to about 63%, about 43% to about 62%, about 44% to about 61%, about 45% to about 60%, or about 50% to about 55%.

The target nucleic acid can include a sequence with GC content of from about 35% to about 70%, about 40% to about 65%, about 43% to about 63%, about 43% to about 62%, about 44% to about 61%, about 45% to about 60%, or about 50% to about 55%.

In some embodiments, the forward internal primer (FIP) 102 can include two regions as shown, which are the F1c region at 5′ end and the F2 region at 3′ end. The F1c region can have a melt temperature of about 62° C., to about 68° C., about 64° C. to about 66° C., or about 65° C. The F2 region can have a melt temperature of about 57° C., to about 63° C., about 59° C. to about 61° C., or about 60° C.

In some embodiments, the backward internal primer (BIP) 104 can include two regions as shown, which are the B1c region at 5′ end and the B2 region at 3′ end. The B1c region can have a melt temperature of about 62° C. to about 68° C., about 64° C. to about 66° C., or about 65° C. The B2 region can have a melt temperature of about 57° C. to about 63° C., about 59° C. to about 61° C., or about 60° C.

The forward outer primer (F3) 103 can include a melt temperature of about 57° C. to about 63° C., about 59° C. to about 61° C., or about 60° C.

The backward outer primer (B3) 105 can include a melt temperature of about 57° C. to about 63° C., about 59° C. to about 61° C., or about 60° C.

The forward loop primer (FL) 108 can include a melt temperature of about 62° C. to about 68° C., about 64° C. to about 66° C., or about 65° C.

The backward loop primer (BL) 110 can include a melt temperature of about 62° C. to about 68° C., about 64° C. to about 66° C., or about 65° C.

An example is provided in the following Table 1:

TABLE 1
Primer Parameters

| | Length | | Melt |
|---|---|---|---|
| Primer Type | (nt) | GC Content | Temperature (° C.) |
| (F1c/B1c) | 20-22 | 45-60% | 64-66 |
| F2/B2 | 18-20 | 45-60% | 59-61 |
| Outer Primers (F3/B3) | 15-25 | 45-60% | 59-61 |
| Loop Primers | 11-25 | 40-65% | 64-66 |
| Target Sequence | N/a | 40-65% | N/A |

There are six primers in Table 1, which are illustrated in FIGS. 1A-1C. The six LAMP primers are the: forward internal primer (FIP) 102 having F1c and F2 regions, backward internal primer (BIP) 104 having B1c and B2 regions, forward outer primer (F3) 103, backward outer primer (B3) 105, loop forward (LF) 108, and loop backward (LB) 110, which are shown in the FIGS. 1A-1C. Applicant points to FIG. 1A for the forward internal primer (FIP) 102 having F1c and F2 regions and the backward internal primer (BIP) 104 having B1c and B2 regions. The corresponding regions shown in Table 1 are combined to form to total FIP or BIP, to get the total length for the primer.

The primers are designed to selectively hybridize to the target nucleic acid and amplicons thereof in accordance with a APE-LAMP protocol. The hybridization can be conducted under hybridization conditions where the primer's base pairs pair up with the corresponding base pairs of the target sequence of the target nucleic acid. The hybridization conditions can include an aqueous medium with a sodium concentration of about 40 mM to about 60 mM, about 45 mM to about 55 mM, about 48 mM to about 52 mM, or about 50 mM. The sodium may be in a salt, such as sodium chloride, and the conditions recited above can be considered for the sodium concentration. The hybridization conditions can include a magnesium concentration of about 0.5 mM to about 12 mM, about 1 mM to about 10 mM, about 2 mM to about 8 mM, or about 4 mM to about 6 mM. The magnesium may be in a salt, such as magnesium, and the conditions recited above can be considered for the magnesium concentration.

The hybridization conditions include the hybridization medium in which hybridization occurs below a certain temperature (e.g., melting temperature) and melting occurs above the melting temperature. The melting temperature can be set as defined herein, such as recited above or in Table 1. The temperature at which the APE-LAMP assay has the reaction for amplification can be a value within the highest ranges shown above, so that all of the hybridized primers can melt. Accordingly, the reaction temperature for the APE-LAMP can preferably be between 55° C. to about 70° C., about 50° C. to about 65° C., about 60° C. to about 62° C., or about 65° C.

Table 2 further illustrates the differences between traditional LAMP and asymmetrical PE-LAMP (APE-LAMP). Notably, the FIP and BIP do not have symmetric concentrations. In this instance, the BIP concentration is 10× lower than the FIP concentration. Moreover, only 1 loop primer is used at 2× the concentration. That is, the forward loop primer is present at twice the amount as typical. These modified concentrations reduce the relative combinations of concatemers, enabling a more specific LAMP reaction for genotyping. However, either the forward loop primer (Loop F) or backward loop primer (Loop B) could be used. Also, the FIP and BIP relative concentrations can be swapped so that the BIP has the higher concentration and the FIP has the lower concentration.

TABLE 2
Comparison of LAMP and APE-LAMP Composition

| | Traditional LAMP | | APE-LAMP | |
|---|---|---|---|---|
| Primer | (Final Conc.) | | (Final Conc.) | |
| FIP (Forward Inner Primer) | 1.6 | uM | 1.6 | uM |
| BIP (Backward Inner Primer) | 1.6 | uM | 0.16 | uM |
| F3 (Forward Outer Primer) | 0.2 | uM | 0.4 | uM |
| B3 (Backward Outer Primer) | 0.2 | uM | 0.4 | uM |
| Loop F (Forward Loop Primer) | 0.4 | uM | 0.8 | uM |
| Loop B (Backward Loop Primer) | 0.4 | uM | 0.0 | uM |

In some embodiments, the amount of the loop primer, whether Loop F or Loop B, can be about 50% of the amount of the inner primer that is present at the higher amount. The Loop F primer is shown at 8 micromoles where the forward inner primer (FIP) is shown at 1.6 micromoles. The Loop F primer may vary from these amounts.

APE-LAMP can be used for SNP genotyping and target sequence identification. The APE-LAMP can be designed to amplify a nucleic acid with an SNP or a nucleic acid having a specific sequence. Thus, the probe sequences can be tailored in order to selectively amplify the target sequence of the target nucleic acid.

In some embodiments, the APE-LAMP can be used for pharmacodiagnostics or pathogens, or for any target nucleic acid for any reason.

In some embodiments, a protocol can be performed to develop LAMP assay primers. These primers can be used in the methods for amplification and detection of the target nucleic acids. The protocols using the primers can be performed for the selected gene variants (target nucleic acids) and demonstrate the presence of the selected gene variants.

The design of the LAMP primers for each gene target can be accomplished using the PrimerExplorer v5 software tool from Eiken. The primers were tested on the bench-top using fluorescence and colorimetric detection to optimize LAMP assay parameters. The optimized parameters can include: reagent, primer, enzyme concentrations, melting temperature, and assay time.

In some embodiments, a parameter for optimization can include the allele specificity of the primer sets. The method for obtaining allele specificity is to run each allele (Allele 1 and Allele 2) with a primer set targeting Allele 1 and Allele 2 (TA1 and TA2). This creates 4 pairs Allele 1—TA1, Allele 1—TA 2, Allele 2—TA 1, and Allele 2—TA 2). The allele specificity is the difference between the detection times for these pairs (Allele 1—TA1)—(Allele 1—TA2) and (Allele 2—TA 2)—(Allele 2—TA1).

Following the development and optimization of the primers sets, an assay that uses colorimetric-based LAMP genotyping can be used for detection of the target nucleic acid. That is, the assay can generate a color when the target nucleic acid is present. For example, the assay for detecting the target nucleic acids can be performed.

In some embodiments, the target nucleic acid can include a polymorphism, such as a polymorphism with pharmacogenetic relevance. Polymorphisms can have clinical consequences regarding the metabolism of specific drugs. For example, The human gene VKORC1 encodes for the enzyme Vitamin K epoxide Reductase Complex subunit 1, primarily made in the liver. Notably, VKORC1 variants as significant predictors of warfarin dose¹⁴. Specifically, rs9934438 minor allele homozygous carriers AA is associated with warfarin resistance and requires the highest dosing, whereas genotype GG was not associated with any resistance¹⁵.

Cytochrome P450 2C19 is an enzyme protein responsible for the metabolism of some pharmaceutical compounds. Variant alleles related to pharmaceutical metabolism are often associated with decreased or enhanced enzymatic activity. Notably, rs4244285 is a SNP in the CYP2C19 gene, encoding the CYP2C19*2 variant, and is the most common reason for the poor metabolism of numerous pharmaceuticals, such as warfarin and anti-depressants^{16,17}. Specifically, the GG and AG genotypes are correlated to poor metabolism, whereas the AA genotype results in normal metabolism¹⁸. Further, the phenotypes suggested for pharmacogenetic variants are termed ultrarapid metabolizers (UM), extensive metabolizers (EM), and poor metabolizers (PM).

Multidrug resistance gene, known as MDR1 or ABCB1, encodes for the production of p-glycoprotein (Pgp), a membrane protein. Often drugs that are developed for the treatment of human diseases are also substrates of Pgp. Subsequently, the degree of expression and the functionality of the MDR1 gene product can directly affect the therapeutic effectiveness. A single nucleotide polymorphism, rs1045642, is responsible for the slower metabolism of certain drugs, including morphine. The CC genotype is associated with a normal metabolizer, whereas CT is associated with a slower metabolizer and TT with a poor metabolizer^{19,20}.

The single nucleotide polymorphisms of interest are listed in Table 3, below, alongside their unique identifiers, beginning with an ‘rs’ prefix. While the exemplified protocol uses three commonly studied variant alleles on the CYP2C19, MDR1, and VKORC1 genes, the present invention can be applied to any target nucleic acid. However, polymorphisms within these three genes are often responsible for the effectiveness of pharmaceuticals, which can be helpful to determine for effective health management.

In some embodiments, the allele-specific primers can be designed by knowing the target gene sequence and selecting a unique sequence or a sequence with a SNP, or the like. The present protocols can be performed with NEB WarmStart® Colorimetric LAMP 2× Master Mix from New England Biolabs. This kit incorporates the required enzymes and reagents for the LAMP assay. Also, the kit includes a colorimetric dye that turns from pink-to-yellow, which color change enables fast and straightforward visual detection. While some embodiments may employ a commercial kit for the LAMP reagents, variations and custom kits for reagent and other components can be fabricated for specific targets. However, the LAMP primers are designed to be unique sequences to detect the SNPs of interest on the target nucleic acids, such as the VKORC1, MDR1, and CYP2C19 genes.

### Experiment

### Protocols for Selecting Primers

The following protocol can be performed for selecting primers: Select a target sequence for LAMP design; and use the parameters and a primer designer to generate F1c/B1c/F2/B2/F3/B3 primers. If selecting for Single Nucleotide Genotyping (SNP) genotyping the loop primers are manually selected: Loop primers are designed such that the SNP resides in the middle or +/−1 from the middle in case of even number primers; Loop primers are created from 11 bp to 17 bp for testing; The loop primers are then evaluated using a qPCR machine or colorimetric evaluation to determine the maximum time gap between the SNP of interest (intended to be amplified) and off-target SNPs.

If selecting for specificity maximization, loop primers comply with the table above. Both forward and reverse loop primers are created. In selected buffer and fluorometric or colorimetric mix, the protocol can be tested with known positive and negatives. In the case of NG, 15 positive NG samples were tested alongside 15 negatives. The primer set with the highest specificity were selected. In this case we tested three primer sets, one targeting the OpA gene and two targeting the PorA gene. In all three cases, we tested the forward and reverse configurations.

### Neisseria-Gonorrhea (NG) Primers:

### Neisseria Gonorrhea Preparation:

Streak indicated NG strain on chocolate agar plates; Grow NG at 36 C and 5% C02 (1-2 days depending on single colony growth); Inoculate Rxn Solution with single colony of NG; and Vortex and spin down. Neisseria Gonorrhea primers are obtained.

The experiment is performed as follows: Make colorimetric reaction mix according to the number of conditions being tested; Vortex and spin down; Pipette 20.5 μL of reaction Solution into each well; Add 2.5 ul of designated loop primer to each well; Pipette 2 μL of NG colony dilution or Nuclease free water (NTCs) into each designated well; Run experiment at 65° C. for 50 minutes; Wait 20 minutes then begin imaging every 5 minutes until reaction is completed.

The analysis can be performed as follows: save extra reaction solution to compare original color to completed reaction; the color change of HNB from purple to light blue indicates a positive. The date can be recorded, such as after 50 minutes, identify each well that had a color change and at what time each well experienced the color change.

The NG Primers targeting the PorA gene were developed using NEB primer design tool, including 2 loop primers. One set of NG primers targeting the OPA gene was also developed. We then tested these primers using 15 strains of NG from the CDC using combinations of the APE-LAMP primer concentrations. The 15 strains are strain numbers 165, 166, 167, 169, 175, 177, 181, 185, 187,189, 192, 195, 198, 200, 202. Two different PorA probe sets were determined, as PorA-27 and PorA-21 as shown below. The OPA was targeted with one set—OPA1.

PorA-27
F3
(SEQ ID NO: 1)
GCGGTACTGACACAGGCT;
B3
(SEQ ID NO: 2)
TCGTAGCGTACGGAAACGT;
FIP
(SEQ ID NO: 3)
ACGCTGTTCAAACGGCCGAC GGCAACCGCCAATCCTTC;
BIP
(SEQ ID NO: 4)
GAAAGACACCGGCGGCTTCA TCGGGTTGGGCAATGTTG;
LF
(SEQ ID NO: 5)
GCGCACTTTACCGAAGC;
LB
(SEQ ID NO: 6)
TCCTTGGGAGGGTAAAAGCTACTAT.
PorA-21
F3
(SEQ ID NO: 7)
GCCATTTGGCAGTTGGAAC;
B3
(SEQ ID NO: 8)
TCGGGTTGGGCAATGTTG;
FIP
(SEQ ID NO: 9)
ACTTTACCGAAGCCGCCTTTCA GCCTACGTCAGCGGTACT;
BIP
(SEQ ID NO: 10)
TCGGCCGTTTGAACAGCGTC AGCTTTTACCCTCCCAAGGA;
LF
(SEQ ID NO: 11)
TTGCCCCAGCCTGTGTC;
LB
(SEQ ID NO: 12)
ACACCGGCGGCTTCAA.
OPA1
F3
(SEQ ID NO: 13)
ACGTCAGACACAGCATCGA;
B3
(SEQ ID NO: 14)
GTTGTGGTAGCGGTAGCC;
FIP
(SEQ ID NO: 15)
GCGCCTGGTGTCCTTAATACCT GGGCTTCTTACCACCAGTAC;
BIP
(SEQ ID NO: 16)
CCATCGCGAAAGCGACAGCA CAGCTTGGGCGTGATGTC
LF
(SEQ ID NO: 17)
ACCCCAGACATTATGCCAG;
LB
(SEQ ID NO: 18)
CGTGGGTCTCGGTGTCAT.

Additionally, primers for APE-LAMP were designed using commercial software to target clinically relevant SNPs in the VKORC1. The primer design for targeting alleles in these three genes is listed below. Notably, the base pair responsible for allele-specific selectivity of the assay is highlighted as bolded, and underlined (e.g., g).

VKORC1 (rs9934438)
FIP
(SEQ ID NO: 19)
AGCCCAGGTGGCTTCTTGGAAAGGGTGGAACCAGGTTAGGAC;
BIP
(SEQ ID NO: 20)
TCCTCTGTTCCCCGACCTCCCCTGACGTGGCCAAAGG;
F3
(SEQ ID NO: 21)
CGAAACCCCTGCCCCA;
B3
(SEQ ID NO: 22)
GGAGGATAGGGTCAGTGACA;
LP(G)
(SEQ ID NO: 32)
TCCAAGGGTCGAT;
LP(A)
(SEQ ID NO: 33)
TCCAAGAGTCGAT;
VKORC1 gBlock
(SEQ ID NO: 25)
TCAAAACAACAACAAACAAAAAACCTGGGGACCTAGGATGTCTTTAA
GGGCCCTTCAGCCTCTAACAGTACTTAAACCAATTAAAAGACTCCTG
TTAGTTACCTCCCCACATCCCCACCCGCAGGACGCTCCGTGATGAGC
AGCTAGCTGGCTGTCAGCTGTGTGGATCACCAAGATTGCATGGAGTG
GGGCTGAGCTGACCAAGGGGGATGAGGGGCGGGGCGGGGGGGGCAGG
GAGGGGGCGGAGCCACTCACCTAACAATAGCTGTAGTGTGTAGAAGA
TGCAACCGAATATGCTGTTGGATTGATTGAGGATGCTGTCCTGTCCC
AGCACATGCTCCACCAGCCCGAAACCCCTGCCCCACCTGGCAGAGGG
GTGGGGGGGGTGGAACCAGGTTAGGACTGTCAACCCAGTGCCTTGGA
CCCTGCCCGAGAAAGGTGATTTCCAAGAAGCCACCTGGGCTATCCTC
TGTTCCCCGACCTCCCATCCTAGTCCAAGgGTCGATGATCTCCTGGC
ACCGGGCACCTTTGGCCACGTCAGGATTCCATGTCACTGACCCTATC
CTCCCCTCTCCCCAGACCAGGCCCGGACGTGGCTACTCCGTAGGCCC
TGCTTTTCATCTTAGACCTTAAGTAAGTCTCTTTTTTTTTTTTTTTT
TTTTTTTTTTTGAGACGGAGTCTCACTCTGGCCCAGGCTGGAGTGCA
GTGGCGCGATCTCGGCTCACTGAAACCTCCGCCTCCCGGGTTCAAGC
GATTCTCCTGCCTTAGCCCCCCCGAGTAGCTGGGATTAAGGCACCCG
CCATAGCGCCCGATTAATTTTTCTATTTTTGGTAGAGACAGGCTTTC
ACCATGTTGGCCAGGCTGGTCAACTCCTGACTTCAAGTGATCCATCC
GCCTCGGCCTCCCAAAGTGCTGGGATTACAGGAGTGGGCCACCGCGC
CCGGCCCTTAAGTAATTCTTAAAATGGCAAGGCTGGTATAACGGTTC
ACTCGGTTTTGCA

Target Wild Type versus Target Mutant (with SNP) amplifications with APE-LAMP were performed with CYP2C9 (Table 6), MDR1 (Table 7), and VKORC (Table 5). The nucleic acids were tested to determine the detection time and change in detection time ACq. Table 2 shows the Wild Type versus the SNP for these genes of interest. Table 2 shows the asymmetric concentrations of the Probe-Enhanced LAMP primers. Table 3 shows example genes and SNPs of interest. Table 4 shows the detection time in minuets and change in detection time from the Wild Type to the Target Mutant. Table 8 shows the APE LAMP primers for CYP2C19 (rs4244285).

TABLE 3
Genes and SNPs of interest.

| | | Location (Chromosome, |
|---|---|---|
| SNP (WT −> MUT) | Gene | Position) |
| rs4244285 (G −> A) | CYP2C19 | (10, 94781859) |
| rs1045642 (T −> C) | MDR1 (aka ABCB1) | (7, 87509329) |
| rs9934438 (G −> A) | VKORC1 | (16, 31093557) |

Table 4 shows the detection time difference between the target wild type and the target mutant, which indicates the time difference is sufficient for distinguishing between the two.

TABLE 4
Detection Time - Asymmetric-LAMP Experimentation.
(Nothing is PE-LAMP, * is APE-LAMP) (Minutes)

| Primer | Target Wild Type | Target Mut | ΔCq |
|---|---|---|---|
| | CYP2C9(C) | CYP2C9(T) | |
| LP(C)13 | 37.5 | 42.5 | 5 |
| LP(T)13 | 47.5 | 35.0 | 12.5 |
| *LP(C)13 | 37.5 | 57.5 | 20 |
| *LP(T)13 | 60+ | 35.0 | 25+ |
| | MDR1(C) | MDR1(T) | |
| LP(C)13 | 19.4 | 36.0 | 16.6 |
| LP(T)13 | 36.6 | 25.2 | 11.4 |
| *LP(C)13 | 23.4 | 40+ | 16.6+ |
| *LP(T)13 | 40+ | 26.7 | 13.3+ |
| | VKORC(G) | VKORC(A) | |
| LP(G)13 | 30.0 | 35.3 | 5.4 |
| LP(A)13 | 40+ | 27.5 | 12.5+ |
| *LP(G)13 | 28.6 | 37.7 | 9.1 |
| *LP(A)13 | 40+ | 27.8 | 12.2+ |
| *LP(G)12-5′ | 37.0 | 60+ | 23+ |
| *LP(G)12-3′ | 57 | 60+ | 3+ |

Table 5 shows the APE-LAMP primer set for VLORC1.

TABLE 5
VKORC1 Primer Design (rs9934438)

| Primer Type | APE-LAMP Primers (5′−>3′) |
|---|---|
| FIP | AGCCCAGGTGGCTTCTTGGAAA-GGGTGGAACCAGGTT |
| | AGGAC |
| | (SEQ ID NO: 19) |
| BIP | TCCTCTGTTCCCCGACCTCC-CCTGACGTGGCCAAAGG |
| | (SEQ ID NO: 20) |
| F3 | CGAAACCCCTGCCCCA |
| | (SEQ ID NO: 21) |
| B3 | GGAGGATAGGGTCAGTGACA |
| | (SEQ ID NO: 22) |
| LP(G)-11 | CCAAGGGTCGA |
| | (SEQ ID NO: 23) |
| LP(A)-11 | CCAAGAGTCGA |
| | (SEQ ID NO: 24) |
| LP(G)-13 | TCCAAGGGTCGAT |
| | (SEQ ID NO: 32) |
| LP(A)-13 | TCCAAGAGTCGAT |
| | (SEQ ID NO: 33) |
| LP(A)-15 | GTCCAAGGGTCGATG |
| | (SEQ ID NO: 34) |
| LP(G)-15 | GTCCAAGAGTCGATG |
| | (SEQ ID NO: 35) |
| LP(G)-3′ | TCC AAG GGT CGA |
| | (SEQ ID NO: 36) |
| LP(G)-5′ | CCA AGG GTC GAT |
| | (SEQ ID NO: 37) |

Table 6 shows the APE-LAMP primer set for CYP2C9 (Rs1799853).

TABLE 6
CYP2C9 (Rs1799853)

FIP:
GTTTCCAAGAATGTCAGTAGAGAAGATAGTAGTCCAGTAAGGTCAGTG
ATATGGAGTAGGGT-(SEQ ID NO: 38)
BIP:
TTCTCAACTCCTCCACAAGGCAGCGGGCTTCCTCTTGAACACGGTCCT
CAATGCTCCTCTTCCCCATCCCAAAATTCCGCAGC-
(SEQ ID NO: 39)
F3: AAGATATGGCCACCCCT-(SEQ ID NO: 40)
B3: AGAAACGCCGGATCTCC-(SEQ ID NO: 41)
LP(C): TTGTGCCAGGA-(SEQ ID NO: 42)
LP(T): TTGTGCCAGGA-(SEQ ID NO: 43)

Table 7 shows the APE-LAMP primer set for MDR1 (rs1045642).

TABLE 7
MDR1 (rs1045642)

FIP: GCAGTCAAACAGGATGGGCTCCGAATGTTCAGTGGCTCCGA
(SEQ ID NO: 44)
BIP: TTGCCTATGGAGACAACAGCCGGTATGTTGGCCTCCTTTGCT
(SEQ ID NO: 45)
F3: CAGCTGCTTGATGGCAAAG
(SEQ ID NO: 46)
B3: CAGTGACTCGATGAAGGCA
(SEQ ID NO: 47)
LP(C): AGAGATCGTGAGG
(SEQ ID NO: 48)
LP(T): AGAGATTGTGAGG
(SEQ ID NO: 49)

Table 8 shows the APE-LAMP primer set for CYP2C19 (rs4244285).

TABLE 8
CYP2C19 (rs4244285)

FIP AGCTCTGGTTGTAATTTAAAACTACAATAAAAATTTCCCCATC
(SEQ ID NO: 50)
BIP TTATTGCATATCTTTTCCCACTATCTTCCATAAAAGCAAGGTT
(SEQ ID NO: 51)
F3 TCAGAGAATTACTACACATG
(SEQ ID NO: 52)
B3 ACTTTCTCCAAAATATCACT
(SEQ ID NO: 53)
LP(G) TTTCCCGGGAAC
(SEQ ID NO: 54)
LP(A) TTTCCCAGGAAC
(SEQ ID NO: 55)

The CYP2C19 gBlock is:
(SEQ ID NO: 56)
CTCCCACTTCTAAAATACTAATTCAATTTCAGAGGCTGCTTGATAGAAA
TCAATATAGCAGGGACTATCTTTGTAGTATCAATCAGGTTGTGCAAACT
CTTTTAACCTATGCTATCATCTCCAAAATGTTAATGTAGTAATTCATAC
CATCTTATATTTCAAGATTGTAGAGAAGAATTGTTGTAAAAAGTAAGAG
AATTAATATAAAGATGCTTTTATACTATCAAAAGCAGGTATAAGTCTAG
GAAATGATTATCATCTTTGATTCTCTTGTCAGAATTTTCTTTCTCAAAT
CTTGTATAATCAGAGAATTACTACACATGTACAATAAAAATTTCCCCAT
CAAGATATACAATATATTTTATTTATATTTATAGTTTTAAATTACAACC
AGAGCTTGGCATATTGTATCTATACCTTTATTAAATGCTTTTAATTTAA
TAAATTATTGTTTTCTCTTAGATATGCAATAATTTTCCCACTATCATTG
ATTATTTCCCGGGAACCCATAACAAATTACTTAAAAACCTTGCTTTTAT
GGAAAGTGATATTTTGGAGAAAGTAAAAGAACACCAAGAATCGATGGAC
ATCAACAACCCTCGGGACTTTATTGATTGCTTCCTGATCAAAATGGAGA
AGGTAAAATGTTAACAAAAGCTTAGTTATGTGACTGCTTGCGTATTTGT
GATTCATTGACTAGTTTTGTGTTTACTACGGATGTTTAACAGGTCAAGG
AGTAATGCTTGAGAAGCATATTTAAGTTTTTATTGTATGCATGAATATC
CAGTAAGCATCATAGAAAATGTAAAATTAAATTGTTAAATAATTAGAAT
ACATAGAAGAAATTGTTTAGATAAATATAATCTATCTGAACAATAAGGA
TGTCAGGATAGGAAAAGCTCTGTTCTGCAGCTTCCAGTGAGATCAGCAC
AGGAGGAACTTAAATTTAAAAGAAAATAAAAAACATCTCCATCAAAAAG
TGAGTGAAGGATATGAACAG
The MDR1 gBlock is:
(SEQ ID NO: 57)
ATTAGCAACCTTACATCTACTACTTTAGTTTCTTTTTGCCATGTAACAT
AACACATTCACAGGATCCAGGGATTAGGACACAGATGTCTTGTGGGAGA
GGGAACATTATTCTGCCTACCACATGCATACATCAGAAACCATGGTTGA
AACACAGGAAACATGACAGTTCCTCAAGGCATACAATTATGACCTTGTT
GGGTTAACCTTCACTATCCAAATTTTAATCACACAAACTTTTCCTTAAT
CTCACAGTAACTTGGCAGTTTCAGTGTAAGAAATAATGATGTTAATTGT
GCTACATTCAAAGTGTGCTGGTCCTGAAGTTGATCTGTGAACTCTTGTT
TTCAGCTGCTTGATGGCAAAGAAATAAAGCGACTGAATGTTCAGTGGCT
CCGAGCACACCTGGGCATCGTGTCCCAGGAGCCCATCCTGTTTGACTGC
AGCATTGCTGAGAACATTGCCTATGGAGACAACAGCCGGGTGGTGTCAC
AGGAAGAGATTGTGAGGGCAGCAAAGGAGGCCAACATACATGCCTTCAT
CGAGTCACTGCCTAATGTAAGTCTCTCTTCAAATAAACAGCCTGGGAGC
ATGTGGCAGCCTCTCTGGCCTATAGTTTGATTTATAAGGGGCTGGTCTC
CCAGAAGTGAAGAGAAATTAGCAACCAAATCACACCCTTACCTGTATAC
AAGCATCTGGCCACACTTCCTGTTTGGGTTAGTTGTTACCTTTACCTGA
TCACCTGACCCTCCTTGTGAGGAAGGGATGAAAGTGTTCGACCACTTCA
GGTTTAGGAGAGAGGAACATTTCTGGGATAGGAGAACTGGAACAATTGT
CTTGATCCAAAGCTATAGGCTTGAGGCTCCACCTTTGTCAGCCTTAGGG
GTAAGTACAATATCTGGAAAGCCTTTCACTTTAAGTCCAAGTACAGAGT
CTGGGTCCCCACCTGCACATGCTGCTTCTGGCCTGCTGAGGAAGTAGGC
ATGACTGTCTCTCCCCATGTC

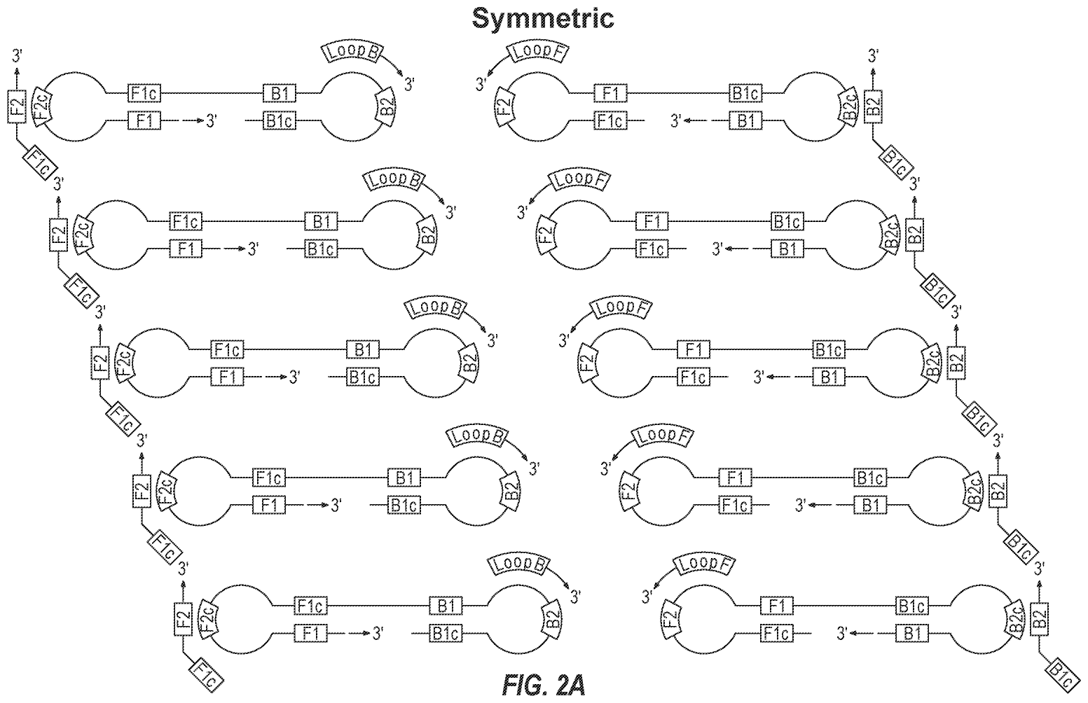

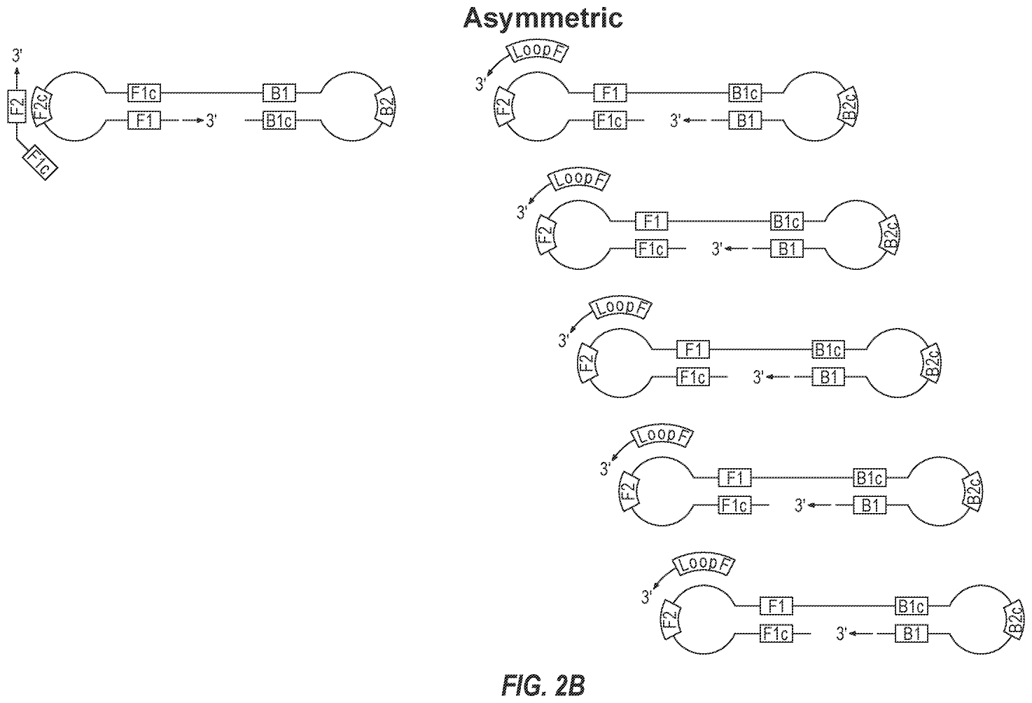

FIG. 2A shows that in normal PE-LAMP the two products are produced in the same amount. FIG. 2B shows that with APE-LAMP the target sequence is prepared with higher specificity. This allows for tailoring the APE-LAMP for genotyping or target sequence selective amplification.

APE-LAMP Initial Testing with MDR1

The APE-LAMP testing was conducted using a high concentration, 1 ng or ˜9.1*10\hat{ }8 copies, of MDR1 gBlocks with the colorimetric LAMP assay at 62° C. A 25 uL solution comprised of NEB WarmStart® Colorimetric LAMP master mix, the MDR1 primers described above at concentrations described in Table 1 with the asymmetric concentrations, and gBlocks was prepared in a centrifuge tube. This LAMP solution was placed in a heatblock at 62° C. for 30 minutes, after which the tubes were removed from the heatblock and observed. The PE-LAMP primer sets targeting both the T and C allele were tested against 1 ng of the MDR1 gBlock (T allele) and a no template control (NTC). Notably, the NTC remained pink, indicating the primers were targeting the gBlock, not due to primer-dimers or other non-specific amplification.

Further, the LAMP mixture containing the matching loop primer LP(T) turned yellow, while the mismatching loop primer LP(C) turned orange. These results indicate that this APE-LAMP strategy can discriminate between alleles. However, ideally, the mismatching primer would remain pink, thereby maximizing the specificity. Therefore, additional experiments were run to increase the Δt between the LP(T) and LP(C) amplifications. Moreover, an experiment identifying the detectable concentrations was performed to understand the limit of detection.

Following this initial experiment using 1 ng gBlock, the APE-LAMP NAAT was conducted using MDR1 gBlock concentrations ranging from 0.01 pg to 100 pg (˜9115 copies to 9.1*10\hat{ }7 copies). The LAMP mixture was held at 62° C. for 20 minutes, then checked every 10 minutes until the yellow color appeared in one or both alleles. Discrimination of allele was possible down to 0.01 pg gBlock (approximately 9115 copies), the lowest quantity tested. Notably, the mixture containing LP(C) remained pinkish while the mixture containing LP(T) turned yellow. Interestingly, the Δt increased as the concentration decreased with little additional time added. This observation is likely because less dumbbell structure was generated as the gBlock concentration is decreased.

Further, this observation may indicate the loop primers start to dominate the reaction at a lower concentration by creating more concatemers rather than the FIP and BIP generating new concatemers.

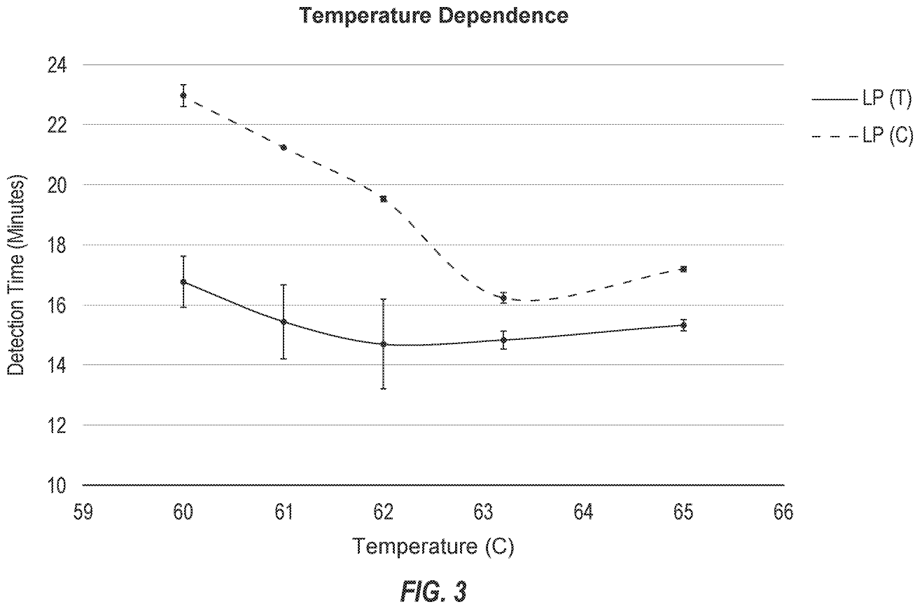

Next, the temperature dependence of APE-LAMP was investigated between 60-65° C. to determine the optimal temperature for allele specificity. A more quantitative approach required using a fluorescent LAMP indicator rather than a colorimetric indicator. The WarmStart LAMP kit E1700S was used for the qLAMP performed in a BIORAD CFX96 using the FAM/SBIR Green channel. The BIORAD qPCR was set to hold temperatures between 60-65° C. for 40 minutes, with the fluorescence quantified every minute of the reaction. The Ct was determined for each condition using a threshold-based measurement. The results for every condition are displayed in FIG. 3, where every reaction was run in duplicate. The blue line (bottom line, LP(T)) represents the detection time for the matching loop primer (T) and the orange line (top line (LP(C)) for the mismatching loop primer (C). Notably, fluorescence-based detection is significantly faster than colorimetric detection. These data conclude the lower end of the temperature range, maximizing the difference in time between the detection of the two-loop primers

The mean and standard deviation of all conditions of this experiment are displayed in Table 9. We conclude from the difference in time between the matched and mismatched loop primers that operating between 60° C. to 65° C. is ideal for these APE-LAMP primers to distinguish between alleles. This section demonstrates the potential for APE-LAMP to differentiate between SNP using the MDR1 APE-LAMP primer sets. Further, we demonstrated the optimal temperature for APE-LAMP with this primer set is 60° C.

TABLE 9
Temperature Dependence of APE-LAMP

| | Ct | Ct | | | |
|---|---|---|---|---|---|
| Temp | (minutes) | (minutes) | Std | Std | Δt |
| (° C.) | LP(T) | LP(C) | LP(T) | LP(C) | (minutes) |
| 65 | 15.3 | 17.2 | 0.084 | 0.181 | 1.9 |
| 63.2 | 14.8 | 16.2 | 0.176 | 0.299 | 1.4 |
| 62 | 14.7 | 19.5 | 0.089 | 1.491 | 4.8 |
| 61 | 15.4 | 21.2 | 0.033 | 1.232 | 5.8 |
| 60 | 16.8 | 23.0 | 0.359 | 0.848 | 6.2 |

APE-LAMP Testing with CYP2C19 and VKORC1

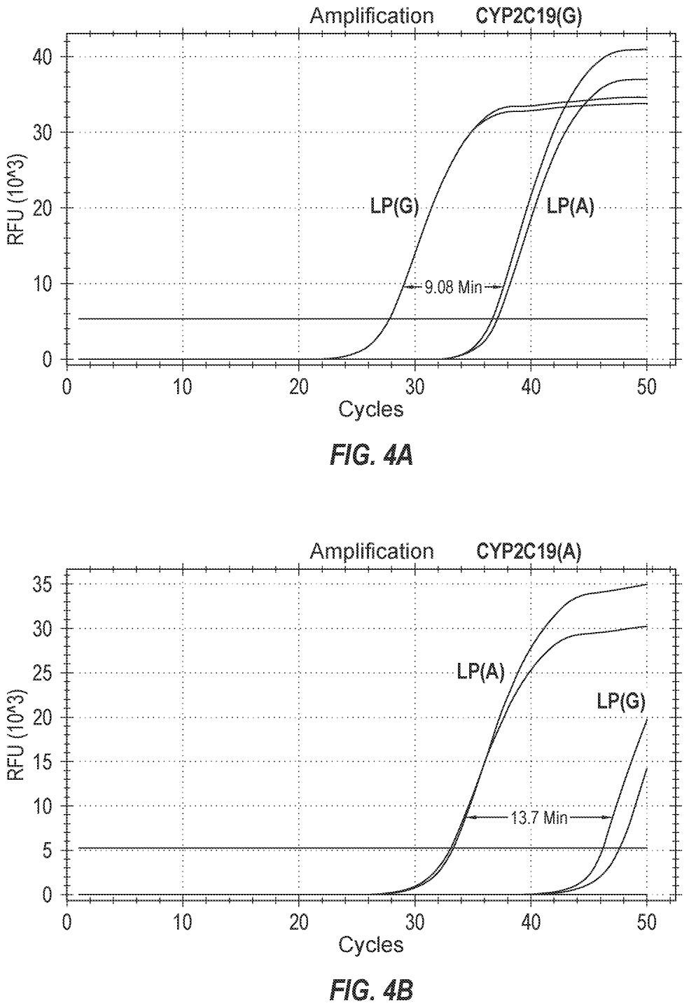

The protocols can include optimization of the allele-specific primer sets using qLAMP analysis. Specifically, the detection time of the APE-LAMP reaction for both A and G alleles when using the primer sets targeting the G allele and A allele were measured using a BioRad qPCR. The temperature was held at 60° C. for 50 minutes, with the same primer concentration as previously used. The results of these experiments are displayed in FIGS. 4A and 4B. These results show a significant difference in detection time between an amplification in which the gBlocks and loop primer match and amplification where the gBlocks and loop primer mismatches. This difference is displayed in FIGS. 4A-4B using an arrow with the time delay listed above. For example, FIG. 4A shows the resulting fluorescent signal from a APE-LAMP using LP(G) and LP(A) with a CYP2C19(G) gBlock. In this case, the LP(G) matches, and the detection time is 27.8 minutes, whereas the LP(A) detection time is 36.9 minutes. The difference between the matching loop primer and mismatching is 9 minutes. The difference between the matching and mismatching loop primers is 13.7 minutes as shown in FIG. 4B. These data demonstrate that genotyping of CYP2C19 is possible due to the differences in amplification time between matching and mismatching loop primers.

The detection time of all combinations from the experiment described above are listed in Table 10. These values are calculated by the BioRad qPCR software using a threshold value, which is displayed as horizontal lines. This threshold value is around 5000 RFU in both FIGS. 4A-4B. The allele-specificity of the CYP2C19 primers is 32.6%, comparable to the allele-specificity of the MDR1 primers described above. The exact detection times are provided in Table 10.

TABLE 10
Detection time for each combination of CYP
template or NTC with each CYP loop primer.

| | LP(G) Detection | LP(A) Detection | Allele |
|---|---|---|---|
| | Time | Time | Specificity |
| Allele | (Minutes) | (Minutes) | (Minutess) |
| CYP2C19(G) | 27.80 | 36.86 | 9.08 |
| CYP2C19(A) | 46.95 | 33.24 | 13.7 |
| NTC | No Amplification | No Amplification | N/A |

The primers were designed to target the SNP on interest in VKORC1 using APE-LAMP. Notably, no software exists to determine the most efficient length or design of the loop primers. Therefore, three different loop primer sets were designed to determine the optimal design as defined by allele specificity. These primer sets are defined in Table 4 as LP-11, LP-13, and LP-15, meaning loop primers of length 11, 13, and 15 base pairs. The sequences of these primers are described in Table 5, and the bold (underlined) base pair indicating the allele specificity.

We performed experiments to optimize the allele specificity based on the loop primer's length. The allele-specificity of loop primers with length 11, 13, and 15 bp results are summarized in Table 11. The 11 base pair long primers did not sufficiently distinguish between the two alleles. Whereas loop primers with length 13 base pairs did sufficiently distinguish between the two alleles with a time gap between the matching loop primer and alleles of 9.4 and 7.9 for VKORC(G) and (A), respectively. The loop primers with length 15 base pairs were equivalent for VKORC(G) but significantly worse for VKORC(A). Therefore, the LP-13 loop primer sets were selected for further analysis. Notably, in all cases, the no template control did not amplify.

TABLE 11
Effect of Loop Primers Length on Allele Specificity

| Loop Primer | Allele Specificity | Allele Specificity |
|---|---|---|
| Length | VKORC(G) | VKORC(A) |
| (Base pairs) | (ΔMinutes) | (ΔMinutes) |
| 11 | 0.5 | 1.1 |
| 13 | 9.4 | 7.9 |
| 15 | 9.3 | 2.06 |

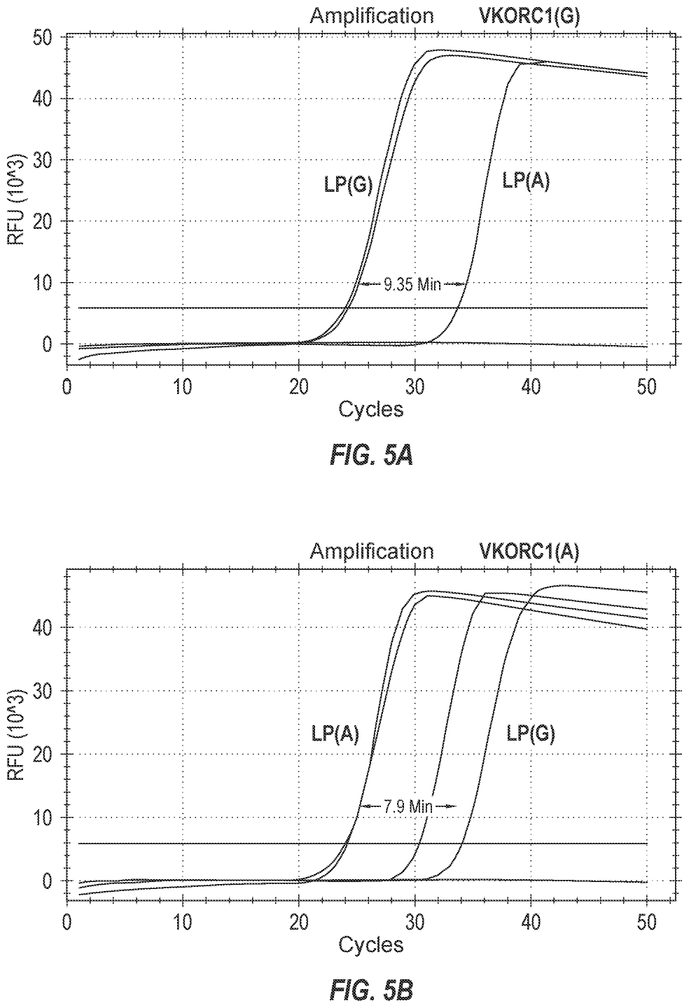

The fluorescence data from these experiments are displayed in FIGS. 5A-5B. Similar to the CYP2C19 experiments, these data show the time gap between matching and mismatching loop primers on a target sequence. VKORC(G) is displayed on the left and VKORC(A) on the right.

The BioRad software analyzed the fluorescence data displayed in FIGS. 5A-5B, with results summarized in Table 12. Again a threshold was established by the BioRad software, and detection times were subsequently determine. The allele-specificity for these primers was 36.7%, similar to the other two primer sets.

TABLE 12
Detection time for each combination of CYP
template or NTC with each CYP loop primer.

| | LP(G) Detection | LP(A) Detection | Allele |
|---|---|---|---|
| | Time | Time | Specificity |
| Allele | (Minutes) | (Minutes) | (Minutess) |
| VKORC1(G) | 25.45 | 34.80 | 9.35 |
| VKORC1(A) | 33.20 | 25.32 | 7.92 |
| NTC | No Amplification | No Amplification | N/A |

After determining the optimal length of the loop primer, the genotyping experiments were performed using colorimetric LAMP. The experiment was performed as described previously for CYP2C19, but using the VKORC primers and gBlocks. These results demonstrate APE-LAMP is capable of differentiating the AA, AG, and GG alleles of the VKORC gene using the primers designed above.

### LAMP-Based Colorimetric Genotyping

Following the optimization and demonstration of APE-LAMP primer sets for allele specificity, we demonstrate genotyping of the three genes of interest using APE-LAMP. In the first experiment gBlocks replicating the rs1045642, C3435T allele, and the C allele was used for this work. The 9000 copies of the C allele were used to represent the CC genotype, 4500 copies of the C allele and 4500 copies of the T allele were used to represent the CT genotype, and 900 copies of the T allele were used to represent the TT genotype. The CT and TT genotypes indicate slower metabolism for some types of drugs. The MDR1 primers described herein were used for this experiment with Loop Primer T, LP(T), targeting the T allele and Loop Primer C, LP(C), targeting the C allele. The experiment demonstrates genotyping by indicating the presence or absence of each allele through the loop primers. A yellow color indicates the CC genotype from the LP(C) mixture and pink color from the LP(T) mixture. The TT genotype is indicated by a yellow color from the LP(T) and a pink color from the LP(C). A yellow color from both the LP(C) and LP(T) indicates the CT genotype. Table 13 gives a representation of each potential outcome.

TABLE 13
APE-LAMP Genotyping

| LP(C)\LP(T) | Yellow | Pink |
|---|---|---|
| Yellow | CT | TT |
| Pink | CC | No Template |

The NTC remains a pink color indicating no contamination for both primer sets. The MDR1 APE-LAMP mixture using LP(T) turned yellow in the presence of TT and (T;C) genotypes, while the CC genotype became a pinkish-orange. The MDR1 APE-LAMP mixture using LP(C) became yellow in the CC and TC genotypes and became orange for the TT genotype. These results are summarized in Table 14. This experiment was successful in indicating that APE-LAMP have the potential for genotyping unknown human samples. However, these results can be improved by modifying the APE-LAMP primer concentrations to ensure the TT genotype is pink during APE-LAMP using LP(C).

TABLE 14
Colorimetric genotyping of MDR1.

| MDR1 PE-LAMP Primers, | | Genotype | | | |
|---|---|---|---|---|---|
| 9000 copies of gBlocks | | TT | (T; C) | CC | NTC |
| Primer | MDR1 | Yellow | Yellow | Light pink | Dark pink |
| Sets | LP(T) | | | | |
| | MDR1 | Orange | Yellow | Yellow | Dark pink |
| | LP(C) | | | | |

Next, we demonstrate genotyping of the CYP2C19 gene using APE-LAMP with the primer described above. CYP2C19 gBlocks replicating the rs424428(A) mutation and the G wildtype allele were used for this work. 9000 copies of the G allele were used to represent the GG genotype, 4500 copies of the G allele and 4500 copies of the A allele were used to represent the AG genotype, and 9000 copies of the A allele were used to represent the TT genotype. The CYP primers described above were used for this experiment with Loop Primer T, LP(G), targeting the G allele, and Loop Primer A, LP(A), targeting the A allele. The experiment conduct demonstrates genotyping by indicating the presence or absence of each allele through the loop primers. The LAMP solutions were prepared as described previously, and the LAMP reaction was performed at 60° C. for 50 minutes. The results of this colorimetric genotyping are summarized in Table 15.

The GG genotype is indicated by the yellow color from the LP(G) mixture and an orange color from the LP(T) mixture. The TT genotype is indicated by an orange color from the LP(T) and a pink color from the LP(G). A yellow color from both the LP(C) and LP(T) indicates the hetrozygous CT genotype. Consistent with the quantitative results, the LP(G) reaction proceeded a bit faster, resulting in a yellow-colored solution for the matching genotypes and an orange-colored solution for the mismatching genotype AA. In comparison, LP(A) proceeded slightly slower, resulting in an orange color for the matching AA and AG and a pink color for GG. While genotyping is still possible under these conditions, the speed of these reactions will be matched in the phase 2 work such that the matching cases will always be yellow. See Table 15.

TABLE 15
Colorimetric genotyping of CYP2C19.

| CYP PE-LAMP Primers, | | Genotype | | | |
|---|---|---|---|---|---|
| 9000 copies of gBlocks | | AA | AG | GG | NTC |
| Primer | MDR1 | Orange | Yellow | Yellow | Pink |
| Sets | LP(G) | | | | |
| | MDR1 | Orange | Orange | Pink | Pink |
| | LP(A) | | | | |

Finally, we demonstrate genotyping VKORC1 using the APE-LAMP primers described previously. The experiment was performed as described previously for CYP2C19, but using the VKORC1 primers and gBlocks. The colors of each conditions are summarized in Table 16. In this case, all the NTC primers remain pink, indicating no contamination. Further, the LP(G) primer set amplifies the AG and GG genotypes, as indicated by the yellow color, whereas the AA genotype is orange. The LP(A) primer set efficiently amplifies the AA and AG genotype while the GG genotype remains an orange color. Similar to the other two genotyping experiments, this assay could be used for successful genotyping in the current state.

TABLE 16
Colorimetric genotyping of VKORCI.

| VKORC1 PE-LAMP Primers, | | Genotype | | | |
|---|---|---|---|---|---|
| 9000 copies of gBlocks | | AA | (T; C) | GG | NTC |
| Primer | VKORC1 | Orange | Yellow | Yellow | Pink |
| Sets | LP(G) | | | | |
| | VKORC1 | Yellow | Yellow | Orange | Pink |
| | LP(A) | | | | |

### Pathogens

The APE-LAMP can be used to detect various types of target nucleic acids, which can be from pathogens, such as food-borne pathogens. The pathogens can be analyzed for unique target nucleic acids for creating the APE-LAMP primers as described herein. The detection of pathogens allows for detection onsite without going into a medical facility. The protocol for the APE-LAMP with a single heating cycle can be performed with simple equipment, such as the cartridges of the incorporated references. The proof of concept can be performed by developing and testing optimized APE-LAMP primer sequences for selected pathogens, STEC Escherichia coli O157:H7 EDL933, Norovirus, and Giardia intestinalis, isolated in water samples. Sensitivity and specificity will be evaluated for multiple concentrations of the pathogens both individually and in combination as well as with potential interfering compounds and/or pathogens.

In the first set of experimentation, we performed comprehensive evaluation of LAMP primers, in conjunction with their corresponding gBlock and non-template controls. Among these outcomes, specific primers were meticulously chosen for further investigation. Notably, Norovirus GI RdRp (Nv G1 RdRp), Norovirus GII RdRp (Nv G2 RdRp), E. coli rfbE, E. coli stx1A Fwd, E. coli stx2A, G. intestinalis 1 Fwd, and G. intestinalis 2 were identified for their potential to cross-react with one another, paving the way for subsequent analyses, as shown in Table 17.

TABLE 17
Norovirus GI

| | Name | Sequence |
|---|---|---|
| Norovirus GI RdRp | | |
| SEQ ID NO: 58 | F3_NvG1-RdRp | CAGACAACAGGTGATGGCC |
| SEQ ID NO: 59 | B3_NvG1-RdRp | GCTTCACCCAGCAGGGATA |
| SEQ ID NO: 60 | FIP_NvG1-RdRp | AATGGAGTTACGGTCGAGCCGTCTCCGGC |
| | | GTACCATCTCA |
| SEQ ID NO: 61 | BIP_NvG1-RdRp | AAGACAGCTTTGGTGGACTCGCTGTGAAT |
| | | GTGGGACCAGGT |
| SEQ ID NO: 62 | LB_NvG1-RdRp | GCCAAACCACGAGACCCAT |
| Norovirus GI capsid | | |
| SEQ ID NO: 63 | F3_NvG1-Cap | AATTCACCTTGGGAGCCT |
| SEQ ID NO: 64 | B3_NvG1-Cap | TCTTGTCACATTTGTCCAAAT |
| SEQ ID NO: 65 | FIP_NvG1-Cap | CAACAGCAGTGCAACTGCTGGACAAAATT |
| | | ATTATTATCCAGGGGT |
| SEQ ID NO: 66 | BIP_NvG1-Cap | CCGCCACAGGTCCATTGAAAAATGGTTGG |
| | | GTTGGAGTG |
| SEQ ID NO: 67 | LB_NvG1-Cap | GCTGTATTAACCTCCGGTACCAGT |
| Norovirus GII RdRp | | |
| SEQ ID NO: 68 | F3_NvG2-RdRp | AATAGCGGCACCGACAAC |
| SEQ ID NO: 69 | B3_NvG2-RdRp | AGGCAAGAGCCAATGTTCAG |
| SEQ ID NO: 70 | FIP_NvG2-RdRp | TCTGATGGGTCCGCAGCCAAGGCTCCAAA |
| | | GCCATAACCT |
| SEQ ID NO: 71 | BIP_NvG2-RdRp | TTGGCGTCATTCGACGCCATATCTGAGCA |
| | | CGTGGGAGG |
| SEQ ID NO: 72 | LF_NvG2-RdRp | CCTCGTCCCAGAGGTCAATAATG |
| SEQ ID NO: 73 | LB_NvG2-RdRp | CATTCACAAAACTGGGAGCCAGA |
| Norovirus GII capsid | | |
| capsid | | |
| SEQ ID NO: 75 | B3_NvG2-Cap | AATGTGCACCTAGCCCCT |
| SEQ ID NO: 76 | FIP_NvG2-Cap | GAGTGGGTGCAGCACTTCTACCTTCACAA |
| | | ATCTCAGCAGGCC |
| SEQ ID NO: 77 | BIP_NvG2-Cap | TTCATGTTGGGATACCCGCTGCTTTCCGG |
| | | GTGAGCAACTTC |
| SEQ ID NO: 78 | LF_NvG2-Cap | GCCCCAGCACAATCTGATGT |

Table 18 contains 3 potential primer sets targeting the G. Intestinalis EF-1a-1 conserved sequences. Again, after the initial development of these primers, NCBI blast was used to determine if these primer sets would potentially amplify other pathogens

TABLE 18
G. Intestinalis EF-1a-1

| | | |
|---|---|---|
| G. int EF-1a-1 | | |
| SEQ ID NO: 79 | F3_G.int- | GCTGGATCTTCTTCGGGTG |
| | EF1a-1 | |
| SEQ ID NO: 80 | B3_G.int- | AGTCCGTCGAGATGCACC |
| | EF1a-1 | |
| SEQ ID NO: 81 | FIP_G.int- | TCGTCGGGGACGTGACGAACATGAC |
| | EF1a-1 | GATGACCTGGGCG |
| SEQ ID NO: 82 | BIP_G.int- | AGGTCCTTGACGGCGAGCCACGAGG |
| | EF1a-1 | AGCTCAAGAAGGC |
| SEQ ID NO: 83 | LF_G.int- | TCGGCTGCAAGAGCTTCAC |
| | EF1a-1 | |
| SEQ ID NO: 84 | LB_G.int- | GACGTTGAAGCCGACGTTGT |
| | EF1a-1 | |
| G. int EF-1a-2 | | |
| SEQ ID NO: 85 | F3_G.int- | TGAGCGTGCGCTTGTC |
| | EF1a-2 | |
| SEQ ID NO: 86 | B3_G.int- | GGACCTCAAGAAGGGCTACG |
| | EF1a-2 | |
| SEQ ID NO: 87 | FIP_G.int- | CACGCCCGTCATCGACTGCCTTCTG |
| | EF1a-2 | GAGGAAGAGCTGGAA |
| SEQ ID NO: 88 | BIP_G.int- | TGGTTCATGACGATGACCTGGGGAC |
| | EF1a-2 | GTGACGAACGACCCG |
| SEQ ID NO: 89 | LB_G.int- | TGAAGCTCTTGCAGCCGAC |
| | EF1a-2 | |
| G. int EF-1a-3 | | |
| SEQ ID NO: 90 | F3_G.int- | CGAGATGCACCACGAGGAG |
| | EF1a-3 | |
| SEQ ID NO: 91 | B3_G.int- | GTAGCCGGGCTGGATCT |
| | EF1a-3 | |
| SEQ ID NO: 92 | FIP_G.int- | ACGTAGCCCTTCTTGAGGTCCTGGG |
| | EF1a-3 | GACAACGTCGGCTTC |
| SEQ ID NO: 93 | BIP_G.int- | GACGTGACGAACGACCCGCTTCGGG |
| | EF1a-3 | TGGTTCATGACGAT |
| SEQ ID NO: 94 | LB_G.int- | AGCTTCACCGCCCAGGT |
| | EF1a-3 | |

Table 19 contains 5 potential primer sets targeting the E. coli rfbE, Eae, fliC, stx1A, and stx2A conserved regions. Again, after the initial development of these primers, NCBI blast was used to determine if these primer sets would potentially amplify other pathogens.

TABLE 19
E. Coli rfbE, Eae, fliC, stx1A

| | | |
|---|---|---|
| SEQ ID | F3_E. coli- | TGGAATGGTTGTCACGAA |
| NO: 95 | rfbE | |
| SEQ ID | B3_E. coli- | AGCAATTTCACGTTTTCGT |
| NO: 96 | rfbE | |
| SEQ ID | FIP_E. coli- | TTGCCTATGTACAGCTAATCCTTGT |
| NO: 97 | rfbE | GACAAAACACTTTATGACCG |
| SEQ ID | BIP_E. coli- | GGCATGACGTTATAGGCTACAATGC |
| NO: 98 | rfbE | TTGTTCTAACTGGGCTAA |
| SEQ ID | LB_E. coli- | AGGATGACAAATATCTGCGCTGC |
| NO: 99 | rfbE | |
| E. coli Eae | | |
| SEQ ID | F3_E. coli- | AGAGGTTAATCTGCAGAGTG |
| NO: 100 | Eae | |
| SEQ ID | B3_E. coli- | AATGAAGACGTTATAGCCCA |
| NO: 101 | Eae | |
| SEQ ID | FIP_E. coli- | ACCTGACCAAATGCCAGCATTGTAA |
| NO: 102 | Eae | TAACTTTGACGGTAGTTCA |
| SEQ ID | BIP_E. coli- | CGGAGCGCGTTACATTGACTACATG |
| NO: 103 | Eae | TTTGCAGGAAGGAA |
| SEQ ID | LB_E. coli- | CCCGCTTTACGGCAAATTTAGG |
| NO: 104 | Eae | |
| E. coli fliC | | |
| SEQ ID | F3_E. coli- | AACAGCGCGAAGGATGAC |
| NO: 105 | fliC | |
| SEQ ID | B3_E. coli- | TCCAGGTCAGAATCGGAGTT |
| NO: 106 | fliC | |
| SEQ ID | FIP_E. coli- | ATACCGTCGTTGGCGTTACGGCAGG |
| NO: 107 | fliC | TCAGGCGATTGCTAA |
| SEQ ID | BIP_E. coli- | GTTGCGCAGACCACCGAAGGTGGCC |
| NO: 108 | fliC | TGAACCGTCAGTT |
| SEQ ID | LF_E. coli- | CCTGAGTCAGGCCTTTAATGTTAGA |
| NO: 109 | fliC | |
| SEQ ID | LB_E. coli- | CGCGCTGTCCGAAATCAACA |
| NO: 110 | fliC | |
| E. coli | | |
| stx1A | | |
| SEQ ID | F3_E. coli- | GAACTGGGGAAGGTTGAG |
| NO: 111 | stxla | |
| SEQ ID | B3_E. coli- | CACGGACTCTTCCATCTG |
| NO: 112 | stxla | |
| SEQ ID | FIP_E. coli- | TCCCAGAATTGCATTAATGCTTCCG |
| NO: 113 | stxla | TCCTGCCTGATTATCATGG |
| SEQ ID | BIP_E. coli- | GCGTGGCATTAATACTGAATTGTCA |
| NO: 114 | stxla | CATAGAAGGAAACTCATCAGAT |
| SEQ ID | LF_E. coli- | CTTCCTACACGAACAGAGTCTTGT |
| NO: 115 | stxla | |
| SEQ ID | LB_E. coli- | ATCATCATGCATCGCGAGTTGC |
| NO: 116 | stxla | |
| E. coli | | |
| stx2A | | |
| SEQ ID | F3_E. coli- | GACAGTGCCTGACGAAAT |
| NO: 117 | stx2a | |
| SEQ ID | B3_E. coli- | ACAGCAGTTATACCACTCTG |
| NO: 118 | stx2a | |
| SEQ ID | FIP_E. coli- | AGAGCAGTTCTGCGTTTTGTCACTC |
| NO: 119 | stx2a | TGTATCTGCCTGAAGC |
| SEQ ID | BIP_E. coli- | CTGGTCATTGTATTACCACTGAACT |
| NO: 120 | stx2a | ACGTTCCGGAATGCAAAT |
| SEQ ID | LB_E. coli- | TGATGAAACCAGTGAGTGACGAC |
| NO: 121 | stx2a | |

The specificity and sensitivity for each primer set is then evaluated. See Table 20 below. In this case, the PorA-27 primer set which uses the forward loop primer and reduced FIP was selected as the best-performing primer set. Here, LB indicates presence of backward loop primer, and LF indicates presence of Forward loop primer. All include reduced FIP.

TABLE 20
Specificity and Sensitivity

| | APE-LAMP | True | False | Sensi- | Speci- |
|---|---|---|---|---|---|
| Gene | Type | Positive | Positive | tivity | ficity |
| OpA1 | LB | 8 | 0 | 53% | 100% |
| OpA1 | LF | 8 | 4 | 53% | 73% |
| PorA1 | LB | 9 | 5 | 60% | 67% |
| PorAl | LF | 8 | 0 | 53% | 100% |
| PorA21 | LB | 13 | 5 | 87% | 67% |
| PorA21 | LF | 10 | 5 | 67% | 67% |
| PorA27 | LB | 12 | 5 | 80% | 67% |
| PorA27 | LF | 14 | 0 | 93% | 100% |

An example reaction mixture for the APE-LAMP can include the following, as shown in the Table below. The reaction mixture includes Colorimetric WarmStart LAMP 2×, which is a low-Tris reaction buffer with all necessary cofactors, Bst 2.0 WarmStart DNA Polymerase, UDG and dUTP, 8 mM MgSO₄, and phenol red for pH indicator (visualization basis) of LAMP products at optimized concentrations for LAMP assay. This Colorimetric WarmStart LAMP 2× can be in the reaction mixture at about 12.5 μL. The LAMP Primer Mix (10×) can be present at about 2.5 μL. The Lamp Primer Mix (10×) can include the primers in the amounts described herein, such as in Table 1 above. The Target DNA (e.g. gBlocks control or target sample) can be present in the reaction mixture about 2 μL. The reaction mixture can include dH₂O at about 8 μL. Total volume of the reaction mixture can be 25 μL.

In some embodiments, the reaction mixture can include the WarmStart LAMP 2× from about 8 μL to about 16 μL, about 10 μL to about 15 μL, about 12 μL to about 13 μL, or about 12.5 μL.

In some embodiments, the reaction mixture can include the LAMP Primer Mix (10×) from about 1 μL to about 4 μL, about 2 μL to about 3 μL, or about 2.5 μL.

In some embodiments, the water can be present at about 4 μL to about 50 μL, about 6 μL to about 30 μL, or about 7 μL to about 25 μL, or about 8 μL to about 15 μL.

In some embodiments, a negative control includes the reaction mixture without any of the LAMP Primer Mix.

In some embodiments, a positive control includes the reaction mixture with a control nucleic acid, such as in the amount as the Target DNA (or RNA). The Table below shows example reaction mixtures with a negative control.

| | |
|---|---|
| DNA | No-Template |
| Target | Control |

| Colorimetric | 12.5 | μl | 12.5 | μl |
|---|---|---|---|---|
| WarmStart LAMP 2X | | | | |
| HNB or Fluorescent | 0.0 | μl | 0.0 | μl |
| dye (50X) | | | | |
| LAMP Primer Mix (10X) | 2.5 | μl | 2.5 | μl |
| Target DNA (e.g. | 2 | μl | 0 | |
| gBlocks or NG sample) | | | | |
| dH2O | 8.0 | μl | 10 | μl |
| Total Volume | 25 | μl | 25 | μl |

### EMBODIMENTS

In some embodiments, an asymmetrical primer set for a target sequence is provided, wherein one primer of a primer pair is provided in a lesser amount compared to the other primer in the primer pair. That is, the primer pair includes an asymmetrical amount of primers with one being present in a higher amount and the other being present in a lower amount. The lower amount can be about 10% of the higher amount. The asymmetrical primer set can include an internal primer pair comprising corresponding internal primers that are the forward internal primer and the backward internal primer. The primer set include an outer primer pair comprising corresponding outer primers that are the forward outer primer and the backward outer primer. The primer set can include at least one loop primer selected from a loop forward primer or a loop backward primer. The loop primer can have a target primer sequence with complementarity with the target sequence. That is, the target sequence can have a unique nucleotide sequence or a unique SNP that can be detected by selective amplification. The loop primer of the primer set includes the sequence with complementarity with the target sequence having the SNP or other unique target sequence. In some aspects, at least one of the primers of a primer pair is present in an amount of at least 50% less than the corresponding primer in its primer pair. In some aspects, the asymmetrical primer pair is the forward internal primer and backward internal primer. In some aspects, the target sequence includes a single nucleotide polymorphism (SNP). In other aspects, the target sequence includes a target sequence that is devoid of a SNP.

In some embodiments, the loop backward primer includes the sequence with complementarity with the target sequence, when the target sequence is on a loop of a hairpin structure (e.g., dumbbell or concatemer) formed from the amplification and generation of the hairpin structure having the loop. In some aspects, the loop forward primer includes the sequence with complementarity with the target sequence. In some aspects, the primer set includes only one loop primer. The only loop primer in the primer set can be the forward loop primer. In other aspects, the loop primer includes only one loop primer being the backward loop primer.

In some embodiments, the loop primer includes the SNP-complementary sequence in a middle region of the loop primer sequence. In some aspects, the loop primer includes the SNP-complementary sequence in a middle region of the loop primer sequence being at least 2 nucleotides from the 5′ end and at least 2 nucleotides from the 3′ end. In some aspects, the loop primer includes the SNP-complementary sequence in a middle region of the loop primer sequence being at least 2, 3, 4, 5, 6, or 7 nucleotides from the 5′ end and at least 2, 3, 4, 5, 6, or 7 nucleotides from the 3′ end.

In an example embodiments, the asymmetrical primer set can include at least one of: the forward internal primer being present at a concentration from about 0.6 μM to about 2.6 μM, from about 1 μM to about 2 μM, from about 1.25 μM to about 1.75 μM, or about 1.6 μM; the backward internal primer being present at a concentration from about 0.6 μM to about 2.6 μM, from about 1 μM to about 2 μM, from about 1.25 μM to about 1.75 μM, or about 1.6 μM; the forward outer primer being present at a concentration from about 0.06 μM to about 1 μM, from about 0.08 μM to about 0.8 μM, from about 0.1 μM to about 0.6 μM, or about 0.2 μM to about 0.5 μM, or about 0.4 μM; the backward outer primer being present at a concentration from about 0.06 μM to about 1 μM, from about 0.08 μM to about 0.8 μM, from about 0.1 μM to about 0.6 μM, or about 0.2 μM to about 0.5 μM, or about 0.4 μM; the loop forward primer being present at a concentration from about 0.1 μM to about 1.6 μM, from about 0.2 μM to about 1.4 μM, from about 0.4 μM to about 1.2 μM, or about 0.4 μM to about 1.0 μM, or about 0.8 μM; or the loop backward primer being present at a concentration from about 0.1 μM to about 1.6 μM, from about 0.2 μM to about 1.4 μM, from about 0.4 μM to about 1.2 μM, or about 0.4 μM to about 1.0 μM, or about 0.8 μM, wherein one of the loop forward primer or loop backward primer is 0 μM (absent or unusably low amount), wherein one of the forward internal primer or backward internal primer is present in an amount of at least 50% less (e.g., 10% less) than the other. In some aspects, the forward internal primer is present at the reduced amount and the loop backward primer is omitted.

In some embodiments, the asymmetrical primer set can include one of the forward internal primer or backward internal primer being present in an amount that is 50% of the other internal primer. In some aspects, one of the forward internal primer or backward internal primer is present in an amount that is 50%, 40%, 30%, 25%, 20%, 15%, 10%, 5%, 2.5%, or 1% of the other internal primer. In some aspects, the backward internal primer is present in an amount that is 50%, 40%, 30%, 25%, 20%, 15%, 10%, 5%, 2.5%, or 1% of the other internal primer. These amounts are relative to each other.

In some embodiments, the target sequence is present in a target nucleic acid of a host having a SNP in a disease-related allele or other target sequence.

In some embodiments, the target sequence is present in a target nucleic acid of a pathogen having a SNP or other target sequence.

In some embodiments the target sequence is present in a target nucleic acid of a pathogen of a sexually transmitted disease.

In some embodiments, the single loop primer is present in an amount that is at least twice as much as a corresponding concentration of a loop mediated isothermal amplification assay with both loop primers.

In some embodiments, a reaction composition can include: the asymmetrical primer set of one of the embodiments; and a target nucleic acid having the target sequence with the asymmetrical probe set. In some aspects, the reaction composition further comprises amplification components, such as a low-Tris reaction buffer with all necessary cofactors, Bst 2.0 WarmStart DNA Polymerase, UDG and dUTP, 8 mM MgSO4, and phenol red for pH indicator (visualization basis) of APE-LAMP products at optimized concentrations for LAMP assay.

In some embodiments, a loop-mediated isothermal amplification assay kit can include: the asymmetrical primer set of one of the embodiments; and a target nucleic acid having the target sequence (e.g., positive control). A negative control nucleic acid having a sequence that is not a target sequence can be provided.

In some embodiments, a method of performing a loop-mediated isothermal amplification (LAMP) assay includes: providing the asymmetrical primer set of one of the embodiments, wherein the asymmetrical primer set is the inner primer set; combining the asymmetrical primer set with a target nucleic acid having the target sequence to form an amplification mixture; heating the amplification mixture to an amplification temperature within an amplification temperature range; maintaining the amplification temperature to amplify the target sequence; and obtaining the amplified target sequence. The amplified target sequence can be visualized, such as with an intercalating dye that becomes visible once amplification crosses a threshold.

In some embodiments, the method can include: obtaining a sample (e.g., biological sample) having the target sequence; splitting the sample into a plurality of aliquots; and providing the asymmetrical primer set to the plurality of aliquots. These aliquots can then be heated with the APE-LAMP reagent to the reaction temperature for the reaction time. Presence of the target sequence can be identified by generation of visible color, such as when enough hybridized nucleic acid for intercalating dye to be visible.

In some embodiments, the biological sample is from a mucosal swab, blood, urine, saliva, or other body fluid.

In some embodiments, the amplified target sequence can be identified by a colorimetric assay with nucleic acid labeling dyes configured for LAMP assays. In some aspects, the method includes: visually analyzing the color of the aliquots after a period of time, with eye or camera; and identifying a color of one or more aliquots that identifies presence of the target sequence in a sample, with observance of color or colorimetric reading from camera. In some aspects, the method can include: obtaining an image of each aliquot with the reagents and sample after the period of time; and analyzing the image for a color that indicate the presence of the target sequence in the sample. In some aspects, the methods include quantifying expression level of the gene having the target sequence, by the level of color generated. The generated color can be compared to a color scale to determine the amount of amplification product of the target sequence.

In some embodiments, the method can include: identifying the target sequence in a biological sample; and determining a physiological condition of a subject that provided the biological sample based on the presence of the target sequence, wherein presence of the target sequence indicates the physiological condition of the subject.

In some embodiments, the method can include: treating the amplification mixture with a fluorescent or colorimetric dye; assaying for fluorescent or colorimetric dye with a camera; and quantitating the amount of the target sequence in the biological sample with a computing device, such as a computer.

In some embodiments, the heating is to a temperature of between 40° C. and 75° C., between 45° C. and 70° C., or between 50° C. and 65° C.

In some embodiments, the method is performed with a wild type target sequence devoid of an SNP and with a mutant target sequence having the NSP. The method can further include determining a time difference for obtaining a visual reading detection time of aliquots of the wild type target sequence devoid of an SNP compared with a visual reading detection time of aliquots of the mutant target sequence having the NSP. In some aspects, the time difference between the visual reading detection time for the wild type target sequence and the visual reading detection time for the mutant target sequence is at least 5 minutes. In some aspects, the time difference between the visual reading detection time for the wild type target sequence and the visual reading detection time for the mutant target sequence is at least 5, 10, 15, 20, 25, 30, 35, 40, 45, 50, 55, 60, 65, 70, or more minutes. In some aspects, the time difference between the visual reading detection time for the wild type target sequence and the visual reading detection time for the mutant target sequence is greater than when the forward internal primer or backward internal primer are present at substantially the same amount. In some aspects, the time difference between the visual reading detection time for the wild type target sequence and the visual reading detection time for the mutant target sequence is greater than when the forward internal primer or backward internal primer are present within 25%, 20%, 15%, 10%, 5%, or 1% of each other.

In some embodiments, the amplification temperature is maintained at less than 60 minutes. In some aspects, the amplification temperature is maintained at less than 45 minutes, or 30 minutes.

In some embodiments, a health condition of a subject is studied by determining the presence or absence of an SNP or other target sequence in a biological sample from the subject. The presence of the target sequence indicates the presence of the related health condition, whether a disease or disorder, or presence or absence of pathogen.

In some aspects, the presence or absence of a pathogen in a subject is studied by determining the presence or absence of a target sequence of a pathogen that is devoid of an SNP.

In some embodiments, a target sequence or an SNP can be used for selective amplification of a gene associated with a condition or disease state of a subject. This can allow for assessing the health, risk of a disease, risk of a poor response to a treatment, or any other information that the presence or absence of a specific nucleotide sequence (e.g., with or without an SNP) under the scope of the present invention.

In some embodiments, a target sequence of a pathogen can be used for selective amplification of the gene associated therewith for identifying the presence or absence of an infection from the pathogen.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.
ADDIN Mendeley Bibliography CSL_BIBLIOGRAPHY 1. Sole C, Arnaiz E, Manterola L, Otaegui D, Lawrie C H. The circulating transcriptome as a source of cancer liquid biopsy biomarkers. In: Seminars in Cancer Biology. Vol 58. Elsevier; 2019:100-108.
2. Kamel H F M, Al-Amodi HSAB. Exploitation of gene expression and cancer biomarkers in paving the path to era of personalized medicine. Genomics Proteomics Bioinformatics. 2017; 15(4):220-235.
3. Dhingra R, Vasan R S. Biomarkers in cardiovascular disease: Statistical assessment and section on key novel heart failure biomarkers. Trends Cardiovasc Med. 2017; 27(2):123-133.
4. Pedrotty D M, Morley M P, Cappola T P. Transcriptomic biomarkers of cardiovascular disease. Prog Cardiovasc Dis. 2012; 55(1):64-69.
5. Tamatam A, Khanum F, Bawa A S. Genetic biomarkers of depression. Indian J Hum Genet. 2012; 18(1):20.
6. Powell T R, McGuffin P, D'Souza U M, et al. Putative transcriptomic biomarkers in the inflammatory cytokine pathway differentiate major depressive disorder patients from control subjects and bipolar disorder patients. PLoS One. 2014; 9(3):e91076.
7. İnal Ç, Tanriöver MD, Erden D D. Novel transcriptional biomarkers for diagnosis and prognosis of sepsis. Acta Medica Cordoba. 2016; 47(1):11-18.
8. Bauer M, Giamarellos-Bourboulis E J, Kortgen A, et al. A transcriptomic biomarker to quantify systemic inflammation in sepsis—a prospective multicenter phase II diagnostic study. EBioMedicine. 2016; 6:114-125.
9. Hockings J K, Castrillon J A, Cheng F. Pharmacogenomics meets precision cardio-oncology: is there synergistic potential? Hum Mol Genet. 2020; 29(R2):R177-R185.
10. Becherer L, Borst N, Bakheit M, Frischmann S, Zengerle R, von Stetten F. Loop-mediated isothermal amplification (LAMP)-review and classification of methods for sequence-specific detection. Anal Methods. 2020; 12(6):717-746.
11. Notomi T, Mori Y, Tomita N, Kanda H. Loop-mediated isothermal amplification (LAMP): principle, features, and future prospects. J Microbiol. 2015; 53(1):1-5.
12. Varona M, Anderson J L. Advances in Mutation Detection Using Loop-Mediated Isothermal Amplification. ACS omega. 2021; 6(5):3463-3469.
13. Ding S, Chen R, Chen G, et al. One-step colorimetric genotyping of single nucleotide polymorphism using probe-enhanced loop-mediated isothermal amplification (PE-LAMP). Theranostics. 2019; 9(13):3723.
14. Pratt V M, Cavallari L H, Del Tredici A L, et al. Recommendations for clinical warfarin genotyping allele selection: a report of the Association for Molecular Pathology and the College of American Pathologists. J Mol Diagnostics. 2020; 22(7):847-859.
15. Al-Mahayri Z N, Al Jaibeji H S, Saab Y, et al. Vkorc1 variants as significant predictors of Warfarin dose in Emiratis. Pharmgenomics Pers Med. 2019; 12:47.
16. Scordo M G, Pengo V, Spina E, Dahl M L, Gusella M, Padrini R. Influence of CYP2C9 and CYP2C19 genetic polymorphisms on warfarin maintenance dose and metabolic clearance. Clin Pharmacol Ther. 2002; 72(6):702-710.
17. Sim S C, Risinger C, Dahl M, et al. A common novel CYP2C19 gene variant causes ultrarapid drug metabolism relevant for the drug response to proton pump inhibitors and antidepressants. Clin Pharmacol Ther. 2006; 79(1):103-113.
18. Saydam F, De\check{g}irmenci İ, Birdane A, et al. The CYP2C19*2 and CYP2C19*17 polymorphisms play a vital role in clopidogrel responsiveness after percutaneous coronary intervention: a pharmacogenomics study. Basic Clin Pharmacol Toxicol. 2017; 121(1):29-36.
19. Kumari R, Lakhan R, Garg R K, Kalita J, Misra U K, Mittal B. Pharmacogenomic association study on the role of drug metabolizing, drug transporters and drug target gene polymorphisms in drug-resistant epilepsy in a north Indian population. Indian J Hum Genet. 2011; 17(Suppl 1):S32.
20. Rojo M, Roco A M, Suarez M, et al. Functionally Significant Coumarin-Related Variant Alleles and Time to Therapeutic Range in Chilean Cardiovascular Patients. Clin Appi Thromb. 2020; 26:1076029620909154.

1. An asymmetrical primer set for a target nucleic acid, comprising:
an internal primer pair comprising corresponding internal primers:
a forward internal primer having a forward hybridizing region on a 3′end and a forward negative hybridizing region on a 5′ end, wherein the forward hybridizing region hybridizes with a forward target sequence of the target nucleic acid and the forward negative hybridizing region does not hybridize with the target nucleic acid; and
a backward internal primer having a backward hybridizing region on a 3′end and a backward negative hybridizing region on a 5′ end, wherein the backward hybridizing region hybridizes with a complementary target sequence of a complementary target nucleic acid and the backward negative hybridizing region does not hybridize with the complementary target nucleic acid, wherein the complementary target nucleic acid is an amplicon of the target nucleic acid or a complementary strand of a double stranded nucleic acid;
an outer primer pair comprising corresponding outer primers:
a forward outer primer having a hybridizing region that hybridizes with the target nucleic acid at an outer region that is 3′ of where the forward hybridizing region hybridizes; and
a backward outer primer having a hybridizing region that hybridizes with the complementary target nucleic acid at an outer region that is 3′ of where the backward hybridizing region hybridizes; and
at least one loop primer selected from a loop forward primer or a loop backward primer, the at least one loop primer having a loop primer sequence with complementarity with a loop target sequence when in a hairpin amplicon or a concatemer amplicon of the target nucleic acid,
wherein at least one of the forward internal primer or backward internal primers is present in an amount of at least 50% less than a corresponding primer in its primer pair.

2. The asymmetrical primer set of claim 1, wherein hybridizing occurs in an aqueous medium with:
a temperature below a melting point of the target sequence with each respective primer;
a sodium concentration of from about 45 mM to about 55 mM;
a magnesium concentration of from about 2 mM to about 8 mM;
a GC content of the target sequence to be about 40% to about 65%; and
a GC content of each primer of the asymmetric primer set to be about 40% to about 60%.

3. The asymmetrical primer set of claim 1, wherein the loop target sequence includes a single nucleotide polymorphism (SNP).

4. The asymmetrical primer set of claim 1, wherein the loop backward primer includes the sequence with complementarity with the loop target sequence.

5. The asymmetrical primer set of claim 1, wherein the loop forward primer includes the sequence with complementarity with the loop target sequence.

6. The asymmetrical primer set of claim 1, wherein the at least one loop primer includes a target-complementary sequence in a middle region that is complementary with a loop target sequence of a loop on a hairpin amplicon or loop on a concatemer amplicon of the target nucleic acid.

7. The asymmetrical primer set of claim 1, wherein the at least one loop primer includes a SNP-complementary sequence in a middle region that is complementary with the SNP sequence on a loop target sequence of a loop on a hairpin amplicon or loop on a concatemer amplicon of the target nucleic acid.

8. The asymmetrical primer set of claim 1, wherein the at least one loop primer includes a SNP-complementary sequence in a middle region, with the SNP complementary sequence being at least 2, 3, 4, 5, 6, or 7 nucleotides from the 5′ end and at least 2, 3, 4, 5, 6, or 7 nucleotides from the 3′ end.

9. The asymmetrical primer set of claim 1, wherein the:
forward inner primer and backward inner primer having a length from about 38 nucleotides to about 42 nucleotides, the forward hybridizing region and backward hybridizing region independently being from about 20 nucleotides to about 22 nucleotides, and forward negative hybridizing region and backward negative hybridizing region being from about 18 nucleotides to about 20 nucleotides;
forward outer primer and backward outer primer having a length from about 15 nucleotides to about 25 nucleotides;
at least one loop primer includes from about 11 nucleotides to about 25 nucleotides.

10. The asymmetrical primer set of claim 1, comprising:
the forward internal primer being present at a concentration from about 0.6 μM to about 2.6 μM, from about 1 μM to about 2 μM, from about 1.25 μM to about 1.75 μM, or about 1.6 μM;
the backward internal primer being present at a concentration from about 0.6 μM to about 2.6 μM, from about 1 μM to about 2 μM, from about 1.25 μM to about 1.75 μM, or about 1.6 μM;
the forward outer primer being present at a concentration from about 0.06 μM to about 1 μM, from about 0.08 μM to about 0.8 μM, from about 0.1 μM to about 0.6 μM, or about 0.2 μM to about 0.5 μM, or about 0.4 μM;
the backward outer primer being present at a concentration from about 0.06 μM to about 1 μM, from about 0.08 μM to about 0.8 μM, from about 0.1 μM to about 0.6 μM, or about 0.2 μM to about 0.5 μM, or about 0.4 μM;
the loop forward primer being present at a concentration from about 0.1 μM to about 1.6 μM, from about 0.2 μM to about 1.4 μM, from about 0.4 μM to about 1.2 μM, or about 0.4 μM to about 1.0 μM, or about 0.8 μM; or
the loop backward primer being present at a concentration from about 0.1 μM to about 1.6 μM, from about 0.2 μM to about 1.4 μM, from about 0.4 μM to about 1.2 μM, or about 0.4 μM to about 1.0 μM, or about 0.8 μM,
wherein one of the loop forward primer or loop backward primer is about 0 μM,
wherein one of the forward internal primer or backward internal primer is present in an amount of at least 50% less than the other.

11. A reaction composition comprising:
the asymmetrical primer set of claim 1; and
a sample having the target nucleic acid, wherein the primer set hybridizes with the target nucleic acid or amplicon thereof.

12. A method of performing an asymmetric loop-mediated isothermal amplification (LAMP) assay, comprising:
providing the asymmetrical primer set of claim 1;
providing a reagent composition having a polymerase;
combining the asymmetrical primer set and reagent composition with a sample having the target nucleic acid to form an amplification mixture;
heating the amplification mixture to an amplification temperature within an amplification temperature range;
maintaining the amplification temperature to amplify the target nucleic acid having the target sequence; and
obtaining an amplified amount of the target nucleic acid having the target sequence.

13. The method of claim 12, further comprising:
obtaining a biological sample having the target nucleic acid;
purifying nucleic acids from the biological sample; and
obtaining a purified nucleic acid sample as the sample that is combined with the asymmetrical primer set.

14. The method of claim 13, wherein the biological sample is from a mucosal swab, blood, urine, saliva, or other body fluid.

15. The method of claim 12, wherein the amplified target nucleic acid can be identified by a colorimetric assay with nucleic acid labeling dye.

16. The method of claim 15, further comprising:
visually analyzing a color generated by the amplified target nucleic acid over a period of time; and
identifying a color that identifies presence of the target nucleic acid in the sample.

17. The method of claim 16, further comprising;
obtaining an image of the amplified target nucleic acid over the period of time; and
analyzing the image for a color that indicates the presence of the target nucleic acid in the sample.

18. The method of claim 12, further comprising:
identifying the target nucleic acid in the sample, wherein the sample is from a biological sample; and
determining a physiological condition of a subject that provided the biological sample based on the presence of the target nucleic acid, wherein presence of the target nucleic acid indicates the physiological condition of the subject.

19. The method of claim 12, further comprising:
treating the amplification mixture with a fluorescent or colorimetric dye;
assaying for fluorescent or colorimetric dye; and
quantitating the amount of the target nucleic acid in the sample.

20. The method of claim 12, wherein the heating is to a temperature of between 40° C. and 75° C., between 45° C. and 70° C., or 50° C. and 65° C.


SEQUENCE LISTING (121 sequences)

SEQ ID NO: 1 - DNA, 18 bp, synthetic construct
  source 1..18: mol_type = other DNA
gcggtactgacacaggct

SEQ ID NO: 2 - DNA, 19 bp, synthetic construct
  source 1..19: mol_type = other DNA
tcgtagcgtacggaaacgt

SEQ ID NO: 3 - DNA, 38 bp, synthetic construct
  source 1..38: mol_type = other DNA
acgctgttcaaacggccgacggcaaccgccaatccttc

SEQ ID NO: 4 - DNA, 38 bp, synthetic construct
  source 1..38: mol_type = other DNA
gaaagacaccggcggcttcatcgggttgggcaatgttg

SEQ ID NO: 5 - DNA, 17 bp, synthetic construct
  source 1..17: mol_type = other DNA
gcgcactttaccgaagc

SEQ ID NO: 6 - DNA, 25 bp, synthetic construct
  source 1..25: mol_type = other DNA
tccttgggagggtaaaagctactat

SEQ ID NO: 7 - DNA, 19 bp, synthetic construct
  source 1..19: mol_type = other DNA
gccatttggcagttggaac

SEQ ID NO: 8 - DNA, 18 bp, synthetic construct
  source 1..18: mol_type = other DNA
tcgggttgggcaatgttg

SEQ ID NO: 9 - DNA, 40 bp, synthetic construct
  source 1..40: mol_type = other DNA
actttaccgaagccgcctttcagcctacgtcagcggtact

SEQ ID NO: 10 - DNA, 40 bp, synthetic construct
  source 1..40: mol_type = other DNA
tcggccgtttgaacagcgtcagcttttaccctcccaagga

SEQ ID NO: 11 - DNA, 17 bp, synthetic construct
  source 1..17: mol_type = other DNA
ttgccccagcctgtgtc

SEQ ID NO: 12 - DNA, 16 bp, synthetic construct
  source 1..16: mol_type = other DNA
acaccggcggcttcaa

SEQ ID NO: 13 - DNA, 19 bp, synthetic construct
  source 1..19: mol_type = other DNA
acgtcagacacagcatcga

SEQ ID NO: 14 - DNA, 18 bp, synthetic construct
  source 1..18: mol_type = other DNA
gttgtggtagcggtagcc

SEQ ID NO: 15 - DNA, 42 bp, synthetic construct
  source 1..42: mol_type = other DNA
gcgcctggtgtccttaatacctgggcttcttaccaccagtac

SEQ ID NO: 16 - DNA, 38 bp, synthetic construct
  source 1..38: mol_type = other DNA
ccatcgcgaaagcgacagcacagcttgggcgtgatgtc

SEQ ID NO: 17 - DNA, 19 bp, synthetic construct
  source 1..19: mol_type = other DNA
accccagacattatgccag

SEQ ID NO: 18 - DNA, 18 bp, synthetic construct
  source 1..18: mol_type = other DNA
cgtgggtctcggtgtcat

SEQ ID NO: 19 - DNA, 42 bp, synthetic construct
  source 1..42: mol_type = other DNA
agcccaggtggcttcttggaaagggtggaaccaggttaggac

SEQ ID NO: 20 - DNA, 37 bp, synthetic construct
  source 1..37: mol_type = other DNA
tcctctgttccccgacctcccctgacgtggccaaagg

SEQ ID NO: 21 - DNA, 16 bp, synthetic construct
  source 1..16: mol_type = other DNA
cgaaacccctgcccca

SEQ ID NO: 22 - DNA, 20 bp, synthetic construct
  source 1..20: mol_type = other DNA
ggaggatagggtcagtgaca

SEQ ID NO: 23 - DNA, 11 bp, synthetic construct
  source 1..11: mol_type = other DNA
ccaagggtcga

SEQ ID NO: 24 - DNA, 11 bp, synthetic construct
  source 1..11: mol_type = other DNA
ccaagagtcga

SEQ ID NO: 25 - DNA, 1001 bp, synthetic construct
  source 1..1001: mol_type = other DNA
tcaaaacaacaacaaacaaaaaacctggggacctaggatgtctttaagggcccttcagcctctaacagtacttaaaccaattaaaagactcctgttagttacctccccacatccccacccgcaggacgctccgtgatgagcagctagctggctgtcagctgtgtggatcaccaagattgcatggagtggggctgagctgaccaagggggatgaggggcggggcggggcgggcagggagggggcggagccactcacctaacaatagctgtagtgtgtagaagatgcaaccgaatatgctgttggattgattgaggatgctgtcctgtcccagcacatgctccaccagcccgaaacccctgccccacctggcagaggggtggggtggggtggaaccaggttaggactgtcaacccagtgccttggaccctgcccgagaaaggtgatttccaagaagccacctgggctatcctctgttccccgacctcccatcctagtccaagggtcgatgatctcctggcaccgggcacctttggccacgtcaggattccatgtcactgaccctatcctcccctctccccagaccaggcccggacgtggctactccgtaggccctgcttttcatcttagaccttaagtaagtctctttttttttttttttttttttttttttgagacggagtctcactctggcccaggctggagtgcagtggcgcgatctcggctcactgaaacctccgcctcccgggttcaagcgattctcctgccttagcccccccgagtagctgggattaaggcacccgccatagcgcccgattaatttttctatttttggtagagacaggctttcaccatgttggccaggctggtcaactcctgacttcaagtgatccatccgcctcggcctcccaaagtgctgggattacaggagtgggccaccgcgcccggcccttaagtaattcttaaaatggcaaggctggtataacggttcactcggttttgca

SEQ ID NO: 26-31 - skipped (6 entries)

SEQ ID NO: 32 - DNA, 13 bp, synthetic construct
  source 1..13: mol_type = other DNA
tccaagggtcgat

SEQ ID NO: 33 - DNA, 13 bp, synthetic construct
  source 1..13: mol_type = other DNA
tccaagagtcgat

SEQ ID NO: 34 - DNA, 15 bp, synthetic construct
  source 1..15: mol_type = other DNA
gtccaagggtcgatg

SEQ ID NO: 35 - DNA, 15 bp, synthetic construct
  source 1..15: mol_type = other DNA
gtccaagagtcgatg

SEQ ID NO: 36 - DNA, 12 bp, synthetic construct
  source 1..12: mol_type = other DNA
tccaagggtcga

SEQ ID NO: 37 - DNA, 12 bp, synthetic construct
  source 1..12: mol_type = other DNA
ccaagggtcgat

SEQ ID NO: 38 - DNA, 62 bp, synthetic construct
  source 1..62: mol_type = other DNA
gtttccaagaatgtcagtagagaagatagtagtccagtaaggtcagtgatatggagtagggt

SEQ ID NO: 39 - DNA, 83 bp, synthetic construct
  source 1..83: mol_type = other DNA
ttctcaactcctccacaaggcagcgggcttcctcttgaacacggtcctcaatgctcctcttccccatcccaaaattccgcagc

SEQ ID NO: 40 - DNA, 17 bp, synthetic construct
  source 1..17: mol_type = other DNA
aagatatggccacccct

SEQ ID NO: 41 - DNA, 17 bp, synthetic construct
  source 1..17: mol_type = other DNA
agaaacgccggatctcc

SEQ ID NO: 42 - DNA, 11 bp, synthetic construct
  source 1..11: mol_type = other DNA
ttgtgccagga

SEQ ID NO: 43 - DNA, 11 bp, synthetic construct
  source 1..11: mol_type = other DNA
ttgtgccagga

SEQ ID NO: 44 - DNA, 41 bp, synthetic construct
  source 1..41: mol_type = other DNA
gcagtcaaacaggatgggctccgaatgttcagtggctccga

SEQ ID NO: 45 - DNA, 42 bp, synthetic construct
  source 1..42: mol_type = other DNA
ttgcctatggagacaacagccggtatgttggcctcctttgct

SEQ ID NO: 46 - DNA, 19 bp, synthetic construct
  source 1..19: mol_type = other DNA
cagctgcttgatggcaaag

SEQ ID NO: 47 - DNA, 19 bp, synthetic construct
  source 1..19: mol_type = other DNA
cagtgactcgatgaaggca

SEQ ID NO: 48 - DNA, 13 bp, synthetic construct
  source 1..13: mol_type = other DNA
agagatcgtgagg

SEQ ID NO: 49 - DNA, 13 bp, synthetic construct
  source 1..13: mol_type = other DNA
agagattgtgagg

SEQ ID NO: 50 - DNA, 43 bp, synthetic construct
  source 1..43: mol_type = other DNA
agctctggttgtaatttaaaactacaataaaaatttccccatc

SEQ ID NO: 51 - DNA, 43 bp, synthetic construct
  source 1..43: mol_type = other DNA
ttattgcatatcttttcccactatcttccataaaagcaaggtt

SEQ ID NO: 52 - DNA, 20 bp, synthetic construct
  source 1..20: mol_type = other DNA
tcagagaattactacacatg

SEQ ID NO: 53 - DNA, 20 bp, synthetic construct
  source 1..20: mol_type = other DNA
actttctccaaaatatcact

SEQ ID NO: 54 - DNA, 12 bp, synthetic construct
  source 1..12: mol_type = other DNA
tttcccgggaac

SEQ ID NO: 55 - DNA, 12 bp, synthetic construct
  source 1..12: mol_type = other DNA
tttcccaggaac

SEQ ID NO: 56 - DNA, 1000 bp, synthetic construct
  source 1..1000: mol_type = other DNA
ctcccacttctaaaatactaattcaatttcagaggctgcttgatagaaatcaatatagcagggactatctttgtagtatcaatcaggttgtgcaaactcttttaacctatgctatcatctccaaaatgttaatgtagtaattcataccatcttatatttcaagattgtagagaagaattgttgtaaaaagtaagagaattaatataaagatgcttttatactatcaaaagcaggtataagtctaggaaatgattatcatctttgattctcttgtcagaattttctttctcaaatcttgtataatcagagaattactacacatgtacaataaaaatttccccatcaagatatacaatatattttatttatatttatagttttaaattacaaccagagcttggcatattgtatctatacctttattaaatgcttttaatttaataaattattgttttctcttagatatgcaataattttcccactatcattgattatttcccgggaacccataacaaattacttaaaaaccttgcttttatggaaagtgatattttggagaaagtaaaagaacaccaagaatcgatggacatcaacaaccctcgggactttattgattgcttcctgatcaaaatggagaaggtaaaatgttaacaaaagcttagttatgtgactgcttgcgtatttgtgattcattgactagttttgtgtttactacggatgtttaacaggtcaaggagtaatgcttgagaagcatatttaagtttttattgtatgcatgaatatccagtaagcatcatagaaaatgtaaaattaaattgttaaataattagaatacatagaagaaattgtttagataaatataatctatctgaacaataaggatgtcaggataggaaaagctctgttctgcagcttccagtgagatcagcacaggaggaacttaaatttaaaagaaaataaaaaacatctccatcaaaaagtgagtgaaggatatgaacag

SEQ ID NO: 57 - DNA, 1001 bp, synthetic construct
  source 1..1001: mol_type = other DNA
attagcaaccttacatctactactttagtttctttttgccatgtaacataacacattcacaggatccagggattaggacacagatgtcttgtgggagagggaacattattctgcctaccacatgcatacatcagaaaccatggttgaaacacaggaaacatgacagttcctcaaggcatacaattatgaccttgttgggttaaccttcactatccaaattttaatcacacaaacttttccttaatctcacagtaacttggcagtttcagtgtaagaaataatgatgttaattgtgctacattcaaagtgtgctggtcctgaagttgatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagattgtgagggcagcaaaggaggccaacatacatgccttcatcgagtcactgcctaatgtaagtctctcttcaaataaacagcctgggagcatgtggcagcctctctggcctatagtttgatttataaggggctggtctcccagaagtgaagagaaattagcaaccaaatcacacccttacctgtatacaagcatctggccacacttcctgtttgggttagttgttacctttacctgatcacctgaccctccttgtgaggaagggatgaaagtgttcgaccacttcaggtttaggagagaggaacatttctgggataggagaactggaacaattgtcttgatccaaagctataggcttgaggctccacctttgtcagccttaggggtaagtacaatatctggaaagcctttcactttaagtccaagtacagagtctgggtccccacctgcacatgctgcttctggcctgctgaggaagtaggcatgactgtctctccccatgtc

SEQ ID NO: 58 - DNA, 19 bp, synthetic construct
  source 1..19: mol_type = other DNA
cagacaacaggtgatggcc

SEQ ID NO: 59 - DNA, 19 bp, synthetic construct
  source 1..19: mol_type = other DNA
gcttcacccagcagggata

SEQ ID NO: 60 - DNA, 40 bp, synthetic construct
  source 1..40: mol_type = other DNA
aatggagttacggtcgagccgtctccggcgtaccatctca

SEQ ID NO: 61 - DNA, 41 bp, synthetic construct
  source 1..41: mol_type = other DNA
aagacagctttggtggactcgctgtgaatgtgggaccaggt

SEQ ID NO: 62 - DNA, 19 bp, synthetic construct
  source 1..19: mol_type = other DNA
gccaaaccacgagacccat

SEQ ID NO: 63 - DNA, 18 bp, synthetic construct
  source 1..18: mol_type = other DNA
aattcaccttgggagcct

SEQ ID NO: 64 - DNA, 21 bp, synthetic construct
  source 1..21: mol_type = other DNA
tcttgtcacatttgtccaaat

SEQ ID NO: 65 - DNA, 45 bp, synthetic construct
  source 1..45: mol_type = other DNA
caacagcagtgcaactgctggacaaaattattattatccaggggt

SEQ ID NO: 66 - DNA, 38 bp, synthetic construct
  source 1..38: mol_type = other DNA
ccgccacaggtccattgaaaaatggttgggttggagtg

SEQ ID NO: 67 - DNA, 24 bp, synthetic construct
  source 1..24: mol_type = other DNA
gctgtattaacctccggtaccagt

SEQ ID NO: 68 - DNA, 18 bp, synthetic construct
  source 1..18: mol_type = other DNA
aatagcggcaccgacaac

SEQ ID NO: 69 - DNA, 20 bp, synthetic construct
  source 1..20: mol_type = other DNA
aggcaagagccaatgttcag

SEQ ID NO: 70 - DNA, 39 bp, synthetic construct
  source 1..39: mol_type = other DNA
tctgatgggtccgcagccaaggctccaaagccataacct

SEQ ID NO: 71 - DNA, 38 bp, synthetic construct
  source 1..38: mol_type = other DNA
ttggcgtcattcgacgccatatctgagcacgtgggagg

SEQ ID NO: 72 - DNA, 23 bp, synthetic construct
  source 1..23: mol_type = other DNA
cctcgtcccagaggtcaataatg

SEQ ID NO: 73 - DNA, 23 bp, synthetic construct
  source 1..23: mol_type = other DNA
cattcacaaaactgggagccaga

SEQ ID NO: 74 - DNA, 20 bp, synthetic construct
  source 1..20: mol_type = other DNA
gcttgcactcaaacagaacc

SEQ ID NO: 75 - DNA, 18 bp, synthetic construct
  source 1..18: mol_type = other DNA
aatgtgcacctagcccct

SEQ ID NO: 76 - DNA, 42 bp, synthetic construct
  source 1..42: mol_type = other DNA
gagtgggtgcagcacttctaccttcacaaatctcagcaggcc

SEQ ID NO: 77 - DNA, 41 bp, synthetic construct
  source 1..41: mol_type = other DNA
ttcatgttgggatacccgctgctttccgggtgagcaacttc

SEQ ID NO: 78 - DNA, 20 bp, synthetic construct
  source 1..20: mol_type = other DNA
gccccagcacaatctgatgt

SEQ ID NO: 79 - DNA, 19 bp, synthetic construct
  source 1..19: mol_type = other DNA
gctggatcttcttcgggtg

SEQ ID NO: 80 - DNA, 18 bp, synthetic construct
  source 1..18: mol_type = other DNA
agtccgtcgagatgcacc

SEQ ID NO: 81 - DNA, 38 bp, synthetic construct
  source 1..38: mol_type = other DNA
tcgtcggggacgtgacgaacatgacgatgacctgggcg

SEQ ID NO: 82 - DNA, 38 bp, synthetic construct
  source 1..38: mol_type = other DNA
aggtccttgacggcgagccacgaggagctcaagaaggc

SEQ ID NO: 83 - DNA, 19 bp, synthetic construct
  source 1..19: mol_type = other DNA
tcggctgcaagagcttcac

SEQ ID NO: 84 - DNA, 20 bp, synthetic construct
  source 1..20: mol_type = other DNA
gacgttgaagccgacgttgt

SEQ ID NO: 85 - DNA, 16 bp, synthetic construct
  source 1..16: mol_type = other DNA
tgagcgtgcgcttgtc

SEQ ID NO: 86 - DNA, 20 bp, synthetic construct
  source 1..20: mol_type = other DNA
ggacctcaagaagggctacg

SEQ ID NO: 87 - DNA, 40 bp, synthetic construct
  source 1..40: mol_type = other DNA
cacgcccgtcatcgactgccttctggaggaagagctggaa

SEQ ID NO: 88 - DNA, 40 bp, synthetic construct
  source 1..40: mol_type = other DNA
tggttcatgacgatgacctggggacgtgacgaacgacccg

SEQ ID NO: 89 - DNA, 19 bp, synthetic construct
  source 1..19: mol_type = other DNA
tgaagctcttgcagccgac

SEQ ID NO: 90 - DNA, 19 bp, synthetic construct
  source 1..19: mol_type = other DNA
cgagatgcaccacgaggag

SEQ ID NO: 91 - DNA, 17 bp, synthetic construct
  source 1..17: mol_type = other DNA
gtagccgggctggatct

SEQ ID NO: 92 - DNA, 40 bp, synthetic construct
  source 1..40: mol_type = other DNA
acgtagcccttcttgaggtcctggggacaacgtcggcttc

SEQ ID NO: 93 - DNA, 39 bp, synthetic construct
  source 1..39: mol_type = other DNA
gacgtgacgaacgacccgcttcgggtggttcatgacgat

SEQ ID NO: 94 - DNA, 17 bp, synthetic construct
  source 1..17: mol_type = other DNA
agcttcaccgcccaggt

SEQ ID NO: 95 - DNA, 18 bp, synthetic construct
  source 1..18: mol_type = other DNA
tggaatggttgtcacgaa

SEQ ID NO: 96 - DNA, 19 bp, synthetic construct
  source 1..19: mol_type = other DNA
agcaatttcacgttttcgt

SEQ ID NO: 97 - DNA, 45 bp, synthetic construct
  source 1..45: mol_type = other DNA
ttgcctatgtacagctaatccttgtgacaaaacactttatgaccg

SEQ ID NO: 98 - DNA, 43 bp, synthetic construct
  source 1..43: mol_type = other DNA
ggcatgacgttataggctacaatgcttgttctaactgggctaa

SEQ ID NO: 99 - DNA, 23 bp, synthetic construct
  source 1..23: mol_type = other DNA
aggatgacaaatatctgcgctgc

SEQ ID NO: 100 - DNA, 20 bp, synthetic construct
  source 1..20: mol_type = other DNA
agaggttaatctgcagagtg

SEQ ID NO: 101 - DNA, 20 bp, synthetic construct
  source 1..20: mol_type = other DNA
aatgaagacgttatagccca

SEQ ID NO: 102 - DNA, 44 bp, synthetic construct
  source 1..44: mol_type = other DNA
acctgaccaaatgccagcattgtaataactttgacggtagttca

SEQ ID NO: 103 - DNA, 39 bp, synthetic construct
  source 1..39: mol_type = other DNA
cggagcgcgttacattgactacatgtttgcaggaaggaa

SEQ ID NO: 104 - DNA, 22 bp, synthetic construct
  source 1..22: mol_type = other DNA
cccgctttacggcaaatttagg

SEQ ID NO: 105 - DNA, 18 bp, synthetic construct
  source 1..18: mol_type = other DNA
aacagcgcgaaggatgac

SEQ ID NO: 106 - DNA, 20 bp, synthetic construct
  source 1..20: mol_type = other DNA
tccaggtcagaatcggagtt

SEQ ID NO: 107 - DNA, 40 bp, synthetic construct
  source 1..40: mol_type = other DNA
ataccgtcgttggcgttacggcaggtcaggcgattgctaa

SEQ ID NO: 108 - DNA, 38 bp, synthetic construct
  source 1..38: mol_type = other DNA
gttgcgcagaccaccgaaggtggcctgaaccgtcagtt

SEQ ID NO: 109 - DNA, 25 bp, synthetic construct
  source 1..25: mol_type = other DNA
cctgagtcaggcctttaatgttaga

SEQ ID NO: 110 - DNA, 20 bp, synthetic construct
  source 1..20: mol_type = other DNA
cgcgctgtccgaaatcaaca

SEQ ID NO: 111 - DNA, 18 bp, synthetic construct
  source 1..18: mol_type = other DNA
gaactggggaaggttgag

SEQ ID NO: 112 - DNA, 18 bp, synthetic construct
  source 1..18: mol_type = other DNA
cacggactcttccatctg

SEQ ID NO: 113 - DNA, 44 bp, synthetic construct
  source 1..44: mol_type = other DNA
tcccagaattgcattaatgcttccgtcctgcctgattatcatgg

SEQ ID NO: 114 - DNA, 47 bp, synthetic construct
  source 1..47: mol_type = other DNA
gcgtggcattaatactgaattgtcacatagaaggaaactcatcagat

SEQ ID NO: 115 - DNA, 24 bp, synthetic construct
  source 1..24: mol_type = other DNA
cttcctacacgaacagagtcttgt

SEQ ID NO: 116 - DNA, 22 bp, synthetic construct
  source 1..22: mol_type = other DNA
atcatcatgcatcgcgagttgc

SEQ ID NO: 117 - DNA, 18 bp, synthetic construct
  source 1..18: mol_type = other DNA
gacagtgcctgacgaaat

SEQ ID NO: 118 - DNA, 20 bp, synthetic construct
  source 1..20: mol_type = other DNA
acagcagttataccactctg

SEQ ID NO: 119 - DNA, 41 bp, synthetic construct
  source 1..41: mol_type = other DNA
agagcagttctgcgttttgtcactctgtatctgcctgaagc

SEQ ID NO: 120 - DNA, 43 bp, synthetic construct
  source 1..43: mol_type = other DNA
ctggtcattgtattaccactgaactacgttccggaatgcaaat

SEQ ID NO: 121 - DNA, 23 bp, synthetic construct
  source 1..23: mol_type = other DNA
tgatgaaaccagtgagtgacgac